(19) United States
(12) Patent Application Publication
(10) Pub. No.: US 2023/0410988 A1
(43) Pub. Date: Dec. 21, 2023

(54) INFORMATION PROCESSING DEVICE AND INFORMATION PROCESSING METHOD

(71) Applicant: PARAMOUNT BED CO., LTD., Tokyo (JP)

(72) Inventors: Takashi ISHIKAWA, Tokyo (JP); Maya HOSHINO, Tokyo (JP)

(73) Assignee: PARAMOUNT BED CO., LTD., Tokyo (JP)

(21) Appl. No.: 18/034,757

(22) PCT Filed: Jun. 29, 2021
(86) PCT No.: PCT/JP2021/024602

§ 371 Date: May 1, 2023

(30) Foreign Application Priority Data
Mar. 15, 2021 (JP) 2021-041033

(51) Int. Cl. G16H 40/20; G06F 16/9535

(52) U.S. Cl. CPC G16H 40/20; G06F 16/9535

(57) ABSTRACT

An information processing device includes a processing unit configured to receive a registration request of a know-how information including information which associates condition information representing a starting condition and assistance information representing an assistance action to be performed if the starting condition is satisfied, and a storage unit configured to store a plurality of the know-how information based on a plurality of the registration requests, wherein the processing unit is configured to output one of the plurality of the know-how information as a search result based on a search request including information to identify either one of the starting condition and the assistance action.

### TECHNICAL FIELD

The present invention relates to an information processing device, an information processing method, etc. This patent application claims the benefit of priority to Japan Patent Application Serial No. 2021-041033, filed on Mar. 15, 2021, which is incorporated by reference herein in its entirety.

### BACKGROUND ART

Traditionally, systems used in medical practice and nursing homes are known. The Patent Document 1 discloses a method for matching service providers to requests from users of long-term care services. The Patent Document 2 discloses a method for determining the route of home care services.

### CITATION LIST

### Patent Literature

Patent Document 1: Japanese Patent Laid-Open No. 2002-007574

Patent Document 2: Japanese Patent Laid-Open No. 2017-191416

### SUMMARY OF THE INVENTION

### Technical Problem

The information processing device and the information processing method that can properly utilize tacit knowledge are provided.

### Solution to a Problem

An information processing device according to the present embodiment includes a processing unit configured to receive a registration request of a know-how information including information which associates condition information representing a starting condition and assistance information representing an assistance action to be performed if the starting condition is satisfied; and a storage unit configured to store a plurality of the know-how information based on a plurality of the registration requests, wherein the processing unit is configured to output one of the plurality of the know-how information as a search result based on a search request including information to identify either one of the starting condition and the assistance action.

### BRIEF DESCRIPTION OF THE DRAWINGS

FIG. 1 shows an example of the information processing system including the information processing device of this embodiment.

FIG. 2 shows a configuration example of a server system.

FIG. 3 shows a configuration example of the terminal device.

FIG. 4 shows to explain the flow of the registration process of the know-how information.

FIG. 5 is an example of the know-how information.

FIG. 6 is a diagram illustrating the flow of process to associate devices, etc. with the know-how information.

FIG. 7 shows a screen example displayed when tagging a correct data.

FIG. 8 is an example of registration information.

FIG. 9 is an example of registration information.

FIG. 10 shows the flow of searching process.

FIG. 11 is an example of list information.

FIG. 12 is a diagram illustrating the flow of process to use the know-how information.

FIG. 13A is an illustration of the first similarity determination process.

FIG. 13B is an illustration of the first similarity determination process.

FIG. 14 is a diagram illustrating the flow of the process for identifying recommended users.

FIG. 15 is a flow chart to explain the second similarity determination process.

FIG. 16 is an example of facility list information.

FIG. 17 shows an example of a user page displaying information about a prescribed user.

FIG. 18 is an example of the know-how information.

FIG. 19A shows an example of a user page displaying information about a prescribed user.

FIG. 19B shows an example of a user page displaying information about a prescribed user.

FIG. 19C shows an example of a user page displaying information about a prescribed user.

FIG. 20 shows an example to associate the know-how information with a plurality of devices.

FIG. 21 is a diagram illustrating the process to identify the recommended supplier based on the know-how information.

FIG. 22 is a screen example that presents recommended suppliers.

FIG. 23 shows a screen example displaying search results for recommended users.

### DESCRIPTION OF EMBODIMENT

Hereafter, the present embodiment will be described with reference to the drawings. In the case of drawings, the same or equivalent elements shall be denoted by the same symbols, and duplicate descriptions shall be omitted. It should be noted that this embodiment described below does not unreasonably limit the contents of the claims. Also, not all of the configurations described in this embodiment are necessarily essential components of this disclosure.

### 1. System Configuration Example

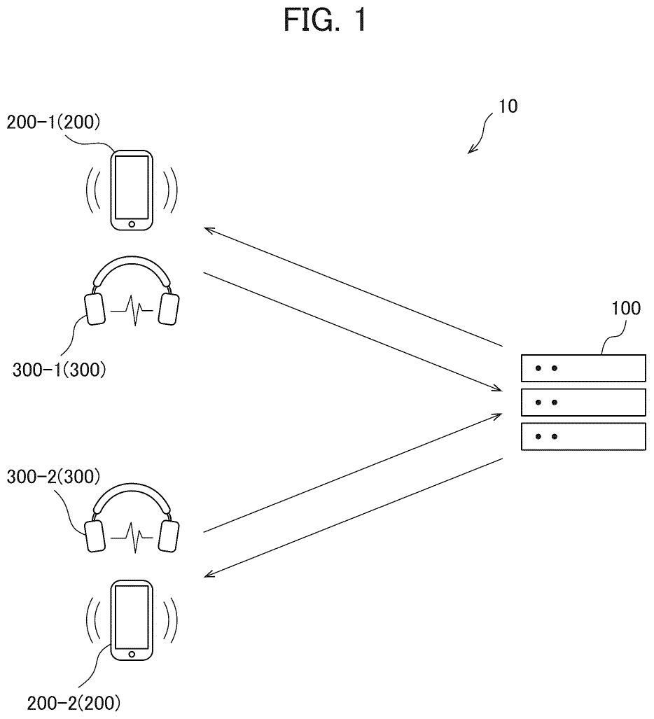

The FIG. 1 is a configuration example of an information processing system 10 including the information processing device according to this embodiment. The information processing system 10 according to the present embodiment provides information to the caregivers so that appropriate assistances can be provided regardless of the skill level of the caregiver by digitizing the “hunch” or “tacit knowledge” of the caregivers about the assistances where the caregivers use the “hunch” or “tacit knowledge”, for example, in nursing facilities. In the following, for the sake of simplicity, “hunch” and “tacit knowledge” are also referred to simply as tacit knowledge. In the following, methods for accumulating and utilizing tacit knowledge about assistance in nursing facilities, hospitals, etc., are described, but this embodiment is not limited to this. For example, in home care where assistance is provided outside of the nursing facilities, tacit knowledge concerning the assistance of caregivers may be accumulated and used, and furthermore, the method of this embodiment provides tacit knowledge of a skilled person, etc. to other users, and is widely applicable to situations where tacit knowledge is used, such as schools, factories, companies, etc.

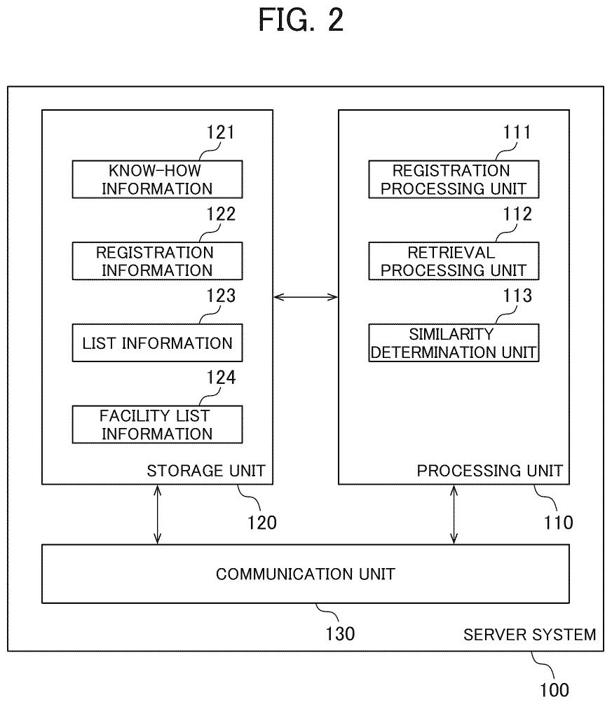

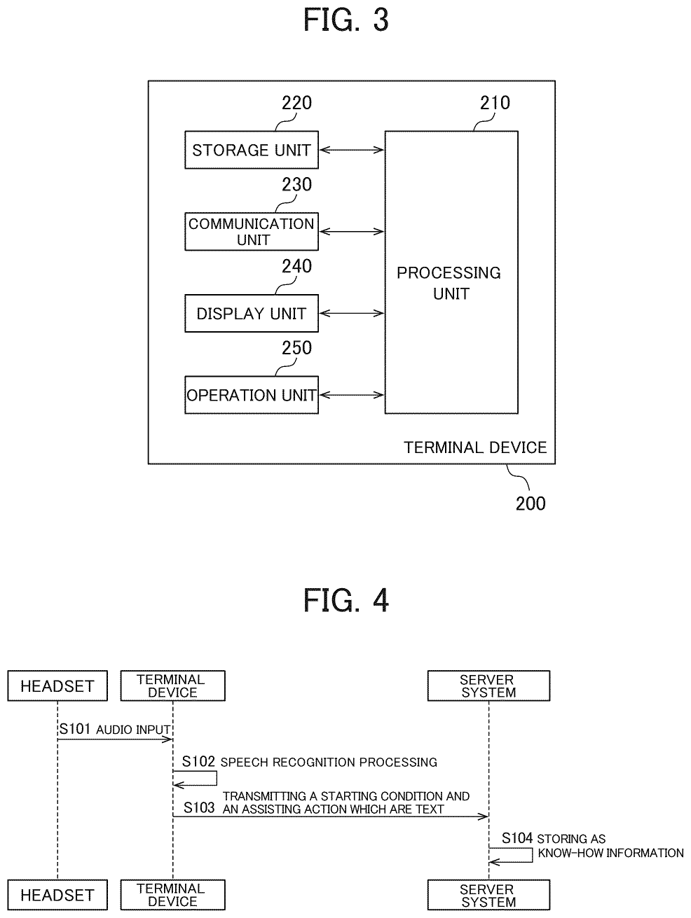

The information processing system 10 shown in the FIG. 1 includes a server system 100, a terminal device 200, and a headset 300. In the FIG. 1, two terminal devices 200-1 and 200-2 are shown as the terminal devices 200. The headset 300-1 and headset 300-2 are shown as the headsets 300. However, the configuration of the information processing system 10 is not limited to the FIG. 1, and various modifications such as omitting a part or adding other configurations are possible. For example, the number of the terminal devices 200 and the headsets 300 may be 3 or more. In addition, modifications such as omitting or adding a configuration can be performed to the FIGS. 2 and 3, which will be described later.

Hereafter, when it is not necessary to distinguish the multiple terminal devices 200 from each other, they are simply denoted as the terminal device 200. Similarly, when it is not necessary to distinguish the multiple headsets 300 from each other, it is simply denoted as the headset 300.

The information processing device of this embodiment corresponds to, for example, the server system 100. However, the method of this embodiment is not limited to this, and the processing of the information processing device may be performed by distributed processing using the server system 100 and other devices. For example, the information processing device of this embodiment may include the server system 100 and the terminal device 200. An example in which the information processing device is the server system 100 is described below.

The server system 100 may, for example, connect and communicate with the terminal device 200 and the headset 300 through the network. The network here is a public communication network such as the Internet, for example, but may also be a LAN (Local Area Network). For example, the terminal device 200 and the headset 300 are devices used by caregivers in the nursing facilities or nurses in the hospitals on duty. The headset 300 is not limited to a device that can be directly connected to the server system 100, but may be a device connected to the server system 100 through the terminal device 200.

The terminal device 200 is not also limited to a device that communicates directly with the server system 100. For example, a relay device (not shown) may be provided in the nursing facilities. The relay device is a device capable of communicating with the server system 100 through a network. The terminal device 200 and the headset 300 may be connected to the relay device using the LAN in the nursing facilities and connected to the server system 100 via the relay device. For example, multiple terminal devices 200 and multiple headsets 300 are expected to be used simultaneously in the nursing facilities. The relay device may perform processing to select the terminal device 200 or the headset 300 to which the information from the server system 100 is to be transmitted. Alternatively, the relay device may be a manager terminal device used by the manager of the nursing facilities and may operate based on the operator's operational input. For example, the information from the server system 100 is displayed on the display of the relay device, and the manager who browses the displayed result may select the destination terminal device 200 or the destination headset 300. In addition, as described above, various modifications can be made to the information processing device of this embodiment, and for example, the above relay device may be included in the information processing device.

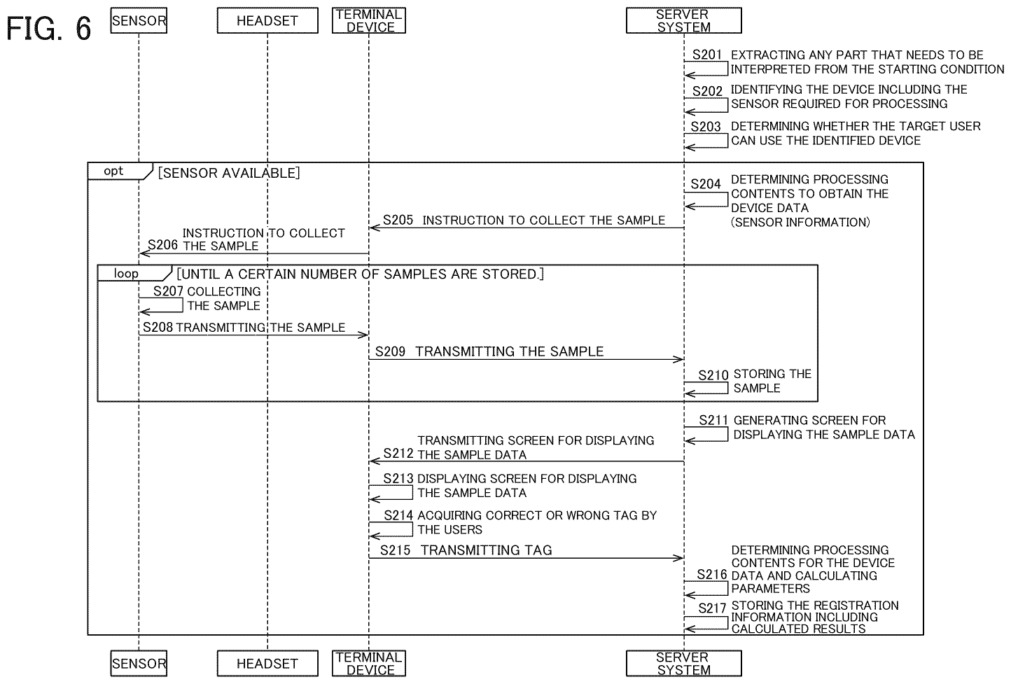

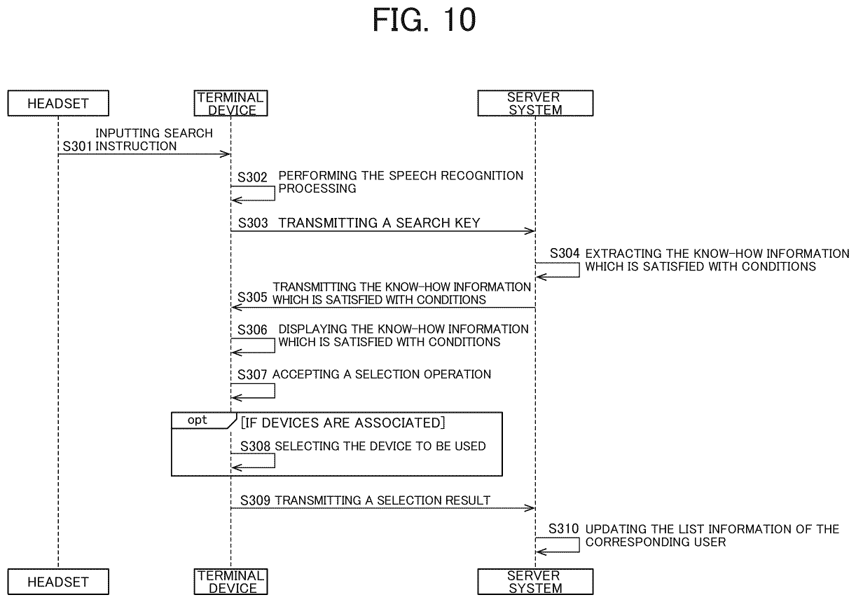

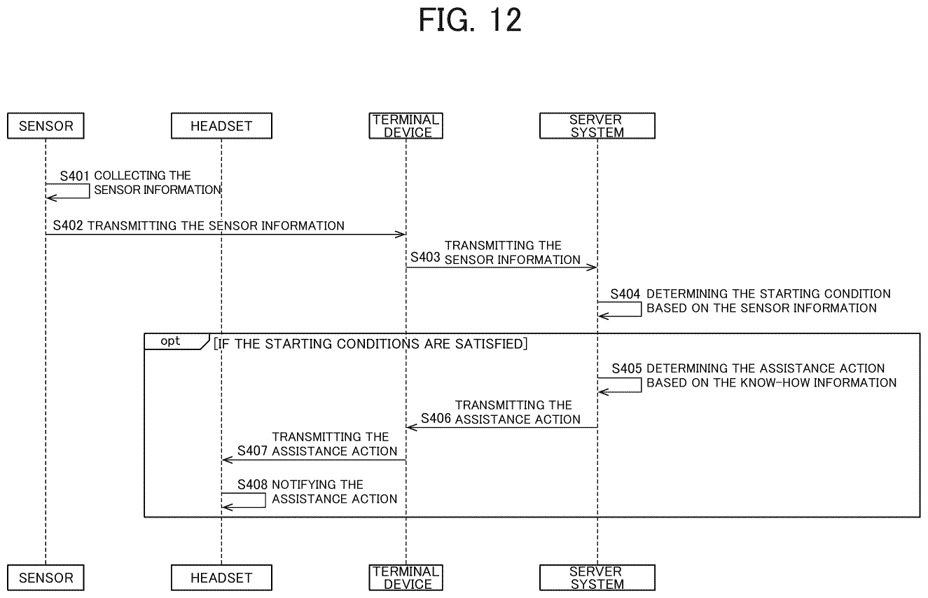

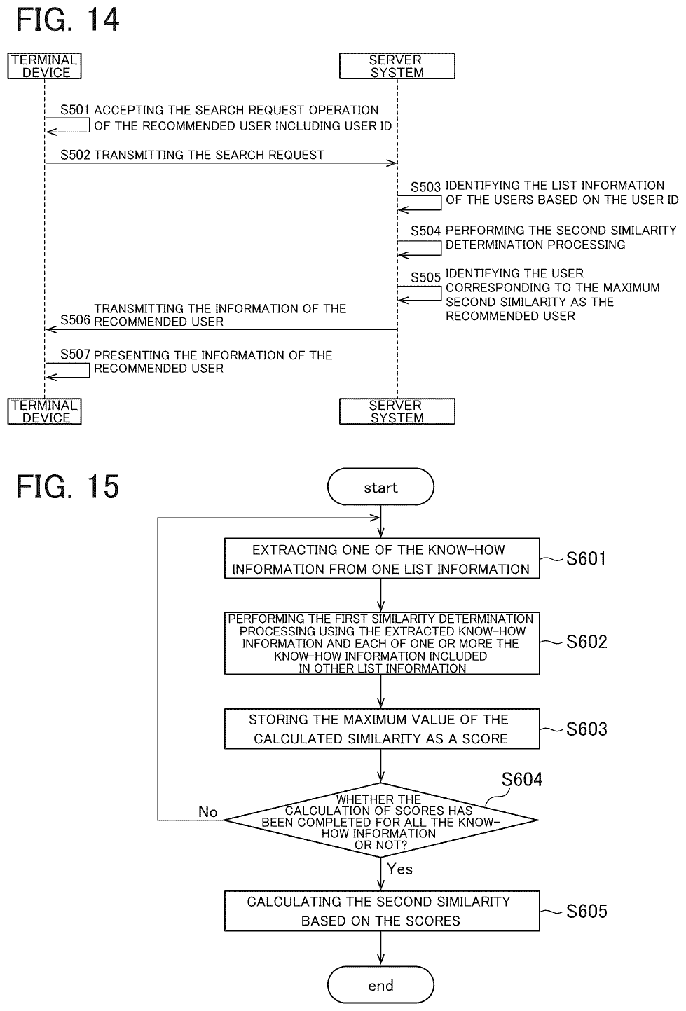

The server system 100 may be a single server or may include multiple servers. For example, the server system 100 may include a data base server and an application server. The database server stores various data described later using the FIG. 2. The application server performs the processing described later using FIG. 4, FIG. 6, FIG. 10, FIG. 12, FIG. 14, FIG. 15, etc. The multiple servers here may be physical servers or virtual servers. If a virtual server is used, the virtual server may be located on one physical server or distributed among multiple physical servers. As described above, the detailed configuration of the server system 100 in this embodiment can be modified in various ways.

The terminal device 200 is a device used to present information provided by the server system 100 to the user and to input information by the user of the terminal device 200. The user in this embodiment may be caregivers who assist an assisted person (a patient, a resident) in, for example, the nursing facilities. Alternatively, the user of the terminal device 200 may be caregivers to visit home who provides visiting care, or a nurse, physical therapist, occupational therapist or speech/language pathologist who assists patients in a hospital, etc. The assistance in the present embodiment represents assistance in daily life which the assisted person can be hard to do by oneself. The Assistance includes a variety of care for the assisted person, such as meal assistance, excretion assistance, transfer and moving assistance. The assistance in this embodiment may be extended to nursing care in the nursing facilities and nursing in hospitals.

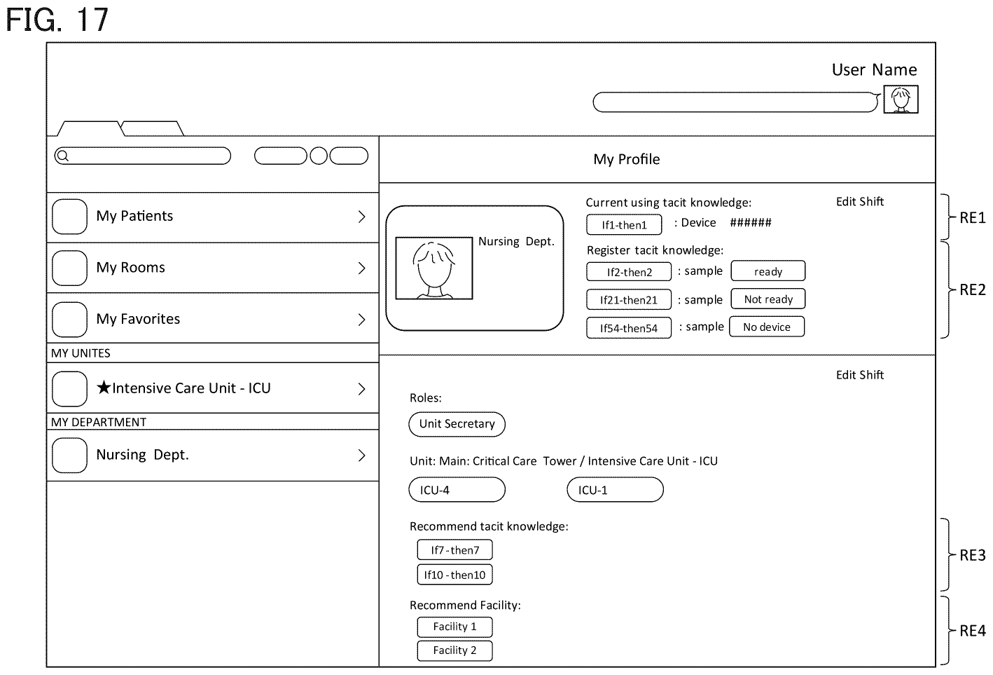

Considering the use of the device in assisting situations, for example, the terminal device 200 is a smartphone or tablet device that is easy to carry. However, the screens described later with reference to the FIG. 17, etc., may be viewed when the caregivers are not assisting, the terminal device 200 may be a PC (Personal Computer) or the like.

The headset 300 also includes earphones or headphones to output sound and a microphone that converts sound into electrical signals and outputs them as audio data. The headset 300 is a device that performs processing for outputting user utterances as audio data and for presenting information from the server system 100 to the user as sound.

For example, a user such as a caregiver rents one terminal device 200 and one headset 300, and uses the terminal device 200 and the headset 300 to communicate with the server system 100. However, the method of this embodiment is not limited to this, and the headset 300 may be omitted from the device used by the user, or other wearable devices may be added. The wearable device here may be a glasses-type device, a wristwatch-type device, or some other form of device.

The FIG. 2 is a block diagram showing a detailed configuration example of the server system 100. The server system 100 includes, for example, a processing unit 110, a storage unit 120, and a communication unit 130. The processing unit 110 accepts a registration request of know-how information 121 including information in which condition information representing a prescribed starting condition is associated with assistance information representing an assistance action to be performed when the starting condition is satisfied. The storage unit 120 stores a plurality of know-how information 121 based on a plurality of registration requests.

The processing unit 110 of this embodiment is composed of the following hardware. The hardware may include at least one of a circuit for processing digital signals and a circuit for processing analog signals. For example, the hardware can consist of one or more circuit devices mounted on a circuit board or one or more circuit elements. One or more circuit devices are for example integrated circuits (ICs), field-programmable gate arrays (FPGAs), etc. One or more circuit elements are, for example, resistors, capacitors, etc.

The processing unit 110 may be realized by the following processors. The server system 100 of the embodiment includes a memory for storing information and a processor operating based on the information stored in the memory. The information is, for example, programs and various kinds of data. The processor includes the hardware. The processor can use a variety of processors such as CPU (Central Processing Unit), GPU (Graphics Processing Unit) and DSP (Digital Signal Processor). The memory may be a semiconductor memory such as SRAM (Static Random Access Memory), DRAM (Dynamic Random Access Memory), or flash memory, or may be a register, a magnetic storage device such as a Hard Disk Drive (HDD), or an optical storage device such as an optical disk device. For example, the memory stores instructions that can be read by a computer, and when the processor performs the instructions, the functions of the processing unit 110 are realized as processing. The instructions here can be those of the instruction set that makes up the program, or they can be instructions that instruct the hardware circuitry of the processor to operate.

The processing unit 110 includes, for example, a registration processing unit 111, a retrieval processing unit 112, and a similarity determination unit 113.

The registration processing unit 111 receives information corresponding to the tacit knowledge of the user and performs processing for storing the information in the storage unit 120. For example, the registration processing unit 111 performs processing for registering the tacit knowledge as know-how information 121, as described later with the reference to the FIG. 4. In addition, the registration processing unit 111 may perform processing to generate and update a registration information 122 that associates devices, processing algorithms, parameters, etc., with the know-how information 121 in order to make the implicit knowledge more accessible, as described later with the reference to the FIG. 6.

The retrieval processing unit 112 performs processing to accept the user's search request and present the search results when there is a user who wants to use the know-how information 121 registered by the registration processing unit 111. Also, when one of the know-how information 121 among the search results is selected by the user, the retrieval processing unit 112 may generate and update a list information 123 that is information that associates the user with the know-how information 121. As a result of this, each user can use the know-how information 121 registered by other caregivers. The list information 123 is more specifically a collection of one or more know-how information 121 which is used by a prescribed user.

The similarity determination unit 113 performs a first similarity determination processing for determining the similarity between the two pieces of know-how information 121. For example, when the retrieval request is obtained, the retrieval processing unit 112 may obtain the result of the first similarity determination processing from the similarity determination unit 113 and decide the know-how information 121 to be presented as the retrieval result based on the result.

The similarity determination unit 113 also performs a second similarity determination processing for determining the similarity between the two list information 123. In addition, when one or more pieces of know-how information 121 associated with a prescribed facility is defined as facility list information 124, the similarity determination unit 113 may perform a third similarity determination processing for determining the similarity between the facility list information 124 and the list information 123. The similarity determination unit 113 performs processing to determine, for example, recommended users and facilities based on the second similarity determination processing and the third similarity determination processing.

The storage unit 120 is a work area of the processing unit 110 and stores various information. The storage unit 120 can be realized by various kinds of memory, and the memory can be a semiconductor memory such as SRAM, DRAM, ROM, or flash memory, a register, a magnetic storage device, or an optical storage device.

The storage unit 120 may store the know-how information 121, the registration information 122, the list information 123 and the facility list information 124. The know-how information 121 consists of information in which the condition information representing the starting condition and the assistance information representing the assist action to be performed when the starting condition is satisfied. The condition information and the assistance information may be, for example, text.

The registration information 122 is information in which a device (sensor) for automating at least the starting condition determination and the detailed processing content of the starting condition determination are associated with the know-how information 121. The list information 123 is a collection of one or more know-how information 121 which are used by a prescribed user. The facility list information 124 is a collection of one or more pieces of know-how information 121 registered by a user belonging to a prescribed facility. The details of each information will be described later. The storage unit 120 may store other information except for this.

The communication unit 130 is an interface for communication through a network and includes, for example, an antenna, a radio frequency (RF) circuit, and a baseband circuit. The communication unit 130 may operate according to control by the processing unit 110 or may include a processor for communication control different from the processing unit 110. The communication unit 130 is an interface for performing communication according to, for example, TCP/IP (Transmission Control Protocol/Internet Protocol). However, various modifications can be made to the detailed communication system.

The FIG. 3 is a block diagram showing a detailed configuration example of the terminal device 200. The terminal device 200 includes, for example, a processing unit 210, a storage unit 220, a communication unit 230, a display unit 240, and an operation unit 250.

The processing unit 210 is composed of the hardware including at least one of a circuit for processing digital signals and a circuit for processing analog signals. The processing unit 210 may also be realized by a processor. It is possible to use a variety of processors such as CPU, GPU, and DSP. By the processor performing the instructions stored in the memory of the terminal device 200, the function of the processing unit 210 is realized as processing.

The storage unit 220 is the work area of the processing unit 210 and is realized by various memories such as SRAM, DRAM, ROM, etc.

The communication unit 230 is an interface for communication via a network, including, for example, the antennas, the RF circuits, and the baseband circuits. The communication unit 230 communicates with the server system 100, for example, via a network.

The display unit 240 is an interface for displaying various information, and may be the liquid crystal display, or an OLED display or any other type of display. The operation unit 250 is an interface that can accept user operations. The operating unit 250 may be a button or the like provided in the terminal device 200. Moreover, the display unit 240 and the operation unit 250 may be a touch panel constructed as one unit.

In addition, the terminal device 200 may include a light emitting unit, a vibration unit, a sound output unit, etc., which are not shown in the FIG. 3. The light-emitting unit is, for example, LED (light emitting diode), which emits light. The vibrating unit is, for example, a motor, which provides notification by vibration. The sound output unit is a speaker, for example, and provides sound notification. The terminal device 200 may also include various sensors such as motion sensors such as accelerometers and gyro sensors, imaging sensors, and Global Positioning System (GPS) sensors.

### 2. Registering Data

Hereafter, the process of registering the know-how information 121 in the storage unit 120 of the server system 100 will be described with the reference to the FIGS. 4 to 9. The following processing is for users who are caregivers or nurses to accumulate their own tacit knowledge in a manner that can be used by others.

### 2.1 Text Registration Process

First, the user inputs a text containing the starting condition of “if xxx is satisfied, then do yyy” and the assisting action to be performed when the starting condition is satisfied. In this way, the server system 100 can store situations and actions that the target user considers important in providing assistance.

The FIG. 4 is a diagram explaining the flow of registration processing of know-how information 121. When this processing is started, the user first makes the above utterance “if xxx is satisfied, do yyy” into the microphone of the headset 300. In the step S101, the headset 300 converts the user's speech into audio data and transmits the audio data to the terminal device 200. As a preceding step of voice input, a recognition processing of a prescribed trigger word may be performed, or operation of an operation unit provided in the headset 300 may be performed.

In the step S102, the terminal device 200 performs a speech recognition processing on the audio data transmitted from the headset 300. The speech recognition processing starts with acoustic analysis, which extracts feature quantities from the audio data. A processing to identify phonemes with similar characteristics using acoustic models is performed for the acoustic analysis results. In addition, speech recognition results are obtained by converting phonemes into words and sentences using phonetic dictionaries and language models. The speech recognition results are the data representing the result of converting the audio data to text data. In the speech recognition processing of the present embodiment, well-known techniques can be widely applied, so further detailed descriptions are omitted.

In the step S103, the terminal device 200 transmits the text data resulting from the speech recognition processing to the server system 100. The text data transmitted in the step S103 is, for example, the text “if xxx is satisfied, do yyy.” Alternatively, in the speech recognition processing, the terminal device 200 may acquire two texts representing a starting condition and an assisting action by detecting words such as “if - - - ” “in the case” and “when”. In the above example, “xxx” is the text representing the starting condition, and “do yyy” is the text representing the assisting action.

In the step S104, the registration processing unit 111 of the server system 100 performs processing to store the know-how information 121 in the storage unit 120 based on the acquired text. For example, the registration processing unit 111 stores information identifying the user who is the sender of the text. The information identifying the user may be the identification information assigned to the headset 300 or the identification information assigned to the terminal device 200. Alternatively, the information identifying the user may be a user ID that uniquely identifies the user. For example, the registration processing unit 111 stores information including the user ID, the text data indicating the starting condition, and the text data indicating an assisting action in a storage unit 120 as the know-how information 121.

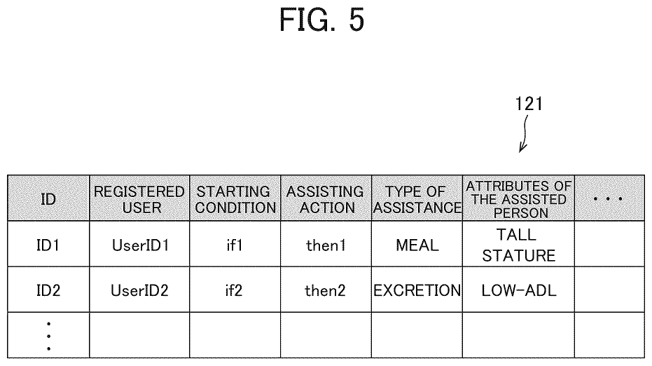

The FIG. 5 is an example of the know-how information 121 stored in the step S104. The ID in the FIG. 5 is information that uniquely identifies the know-how information 121. If the know-how information 121 can be uniquely identified by the set of the starting conditions and the assisting actions, the ID may be omitted. The registered user is a user ID or the like representing a user who has registered the target know-how information 121. The starting conditions and the assisting actions are text data based on user input as described above. The condition information representing the starting condition is not limited to text data only and may include audio data or the result of the speech recognition processing. The result of speech recognition processing is, for example, the result of morpheme analysis and information that associates a morpheme with a part of speech. The same is true for assistance information representing the assisting actions, which may include audio data or the results of speech recognition processing.

Also, as shown in the FIG. 5, the registration processing unit 111 may perform processing to register information other than the above as know-how information 121. For example, the know-how information 121 may include information identifying the situation in which the assisting action is performed. For example, the know-how information 121 includes information indicating which type of assistance is performed, among various types of assistance such as the meal assistance, the excretion assistance, the transferring or moving assistance, etc. The know-how information 121 may also include information representing the attributes of the assisted person to whom the user is assisting. The attributes here include information such as the age, the sex, the height, the weight, the medical history and the medication history of the assisted person. The know-how information 121 may also include physical evaluation data representing the physical evaluation of the assisted person. The physical assessment data includes information such as the evaluation rating of ADL (Activities of Daily Living), the rehabilitation history, the falling risk and the pressure ulcer risk. In this way, it is possible to memorize that the know-how information 121 representing tacit knowledge should be used for what kind of assistance and what kind of the assisted person.

Additional information such as the type of assistance, the attributes of the assisted person, and physical evaluation data may be entered voluntarily by the user. For example, the user makes an utterance including the words “meal,” “tall stature,” etc., in addition to the utterance “After xxx, do yyy.” Alternatively the user may also make a series of utterances including additional information, such as “do yyy if a tall patient do xxx during a meal.”

Also, the server system 100 may transmit the questions such as “what situations do you use it?,” or “What kind of the assisted person should it be used for?” to the headset 300. The above additional information is acquired when the user answers the questions by voice and the text representing the answer result is transmitted to the server system 100.

Thus, in this embodiment, the tacit knowledge is accumulated in the storage unit 120 of the server system 100 as the know-how information 121 including the text. Since the user only needs to use the headset 300 to tweet the starting condition and the assisting actions that the user considers as important actions during the assisting process, so that complicated operation input is not required. This makes it easier to collect large amounts of tacit knowledge used in the nursing facilities and the medical facilities.

In addition, if the collection of know-how information 121 has progressed, the server system 100 may perform an analysis processing of the know-how information 121. For example, the processing unit 110 may perform processing to map each know-how information 121 on the feature space by obtaining feature quantities based on each know-how information 121. For example, the processing unit 110 can estimate particularly important information among tacit knowledge by finding a dense area in the feature space.

Although the FIG. 4 shows an example in which the speech recognition processing is performed in the terminal device 200, it is not limited to this. For example, the terminal device 200 may transmit audio data received from the headset 300 to the server system 100. In this case, the processing unit 110 of the server system 100 may perform the speech recognition processing. The speech recognition processing may also be performed on an external speech recognition server.

The user input may also be done using text rather than audio. For example, the terminal device 200 may acquire text data such as “if xxx is satisfied, then do yyy” by accepting a character input operation by the user. The processing after acquiring the text data is the same as the example in the FIG. 4.

### 2.2 Association to Device

Assume that by performing the processing shown in the FIG. 4, the know-how information 121 including, for example, the starting condition of “If the face of the assisted person starts to wobble during a meal” and an assisting action of “Stop serving the meal” is registered. These texts are meaningful information because they can provide reminders in the meal assistance.

However, this method is not limited to a method to accumulate tacit knowledge as text data, may use to associate further information. For example, the registration processing unit 111 associates information for automatically determining the starting condition with the know-how information 121. In this way, the server system 100 can automatically determine whether the starting condition “the face of the assisted person starts to wobble” is satisfied. As a result, variations in determination of each user can be suppressed, so that a less skilled user can perform the same actions as a more skilled user.

More specifically, in the present embodiment, the information identifying a device having a sensor for data collection, and the information identifying detailed processing content for sensor information from the sensor, etc., may be associated with the know-how information 121. Hereafter, the explanation will be described with the reference to the FIGS. 6 to 8.

The FIG. 6 illustrates the flow of processing to collect information for automation. First, in the step S201, the registration processing unit 111 extracts any part that needs to be interpreted from the condition information representing the starting condition. Specifically, the part that needs to be interpreted is the part that is subject to automatic determination by the server system 100, such as a text data representing the movement of the assisted person, the condition of the assisted person, the environment of the assisted person, etc.

For example, if the starting condition is “If my face of the assisted person starts to wobble during a meal,” the text “Wobble” will represent movement of the assisted person to be detected in the determination of the starting condition. The part of “face” is the information that identifies the part of the body where movement is detected. Therefore, the registration processing unit 111 extracts the part “the face starts to wobble” from the part “the face starts to wobble during the meal” as a part requiring interpretation.

Similarly, in the case that the know-how information 121 which associate the starting condition of “if the assisted person eats only a little meal from a spoon” with the assisting actions of “changing a position that is easy to eat”, “eats only a little meal” represents the direct action of the assisted person. In this case, part of the “meal” is used for determination because that part represents what is to be eaten. “Little” also provides a metric of quantity and is available for determination. Further, the part of “the spoon” also identifies where the meal is located and can be used for determination. Therefore, the registration processing unit 11 extracts, for example, the text “the assisted person eats only a little meal from a spoon” as a part that needs interpretation.

As described above, the registration processing unit 111 may identify the parts that need to be interpreted, for example, by performing morphological analysis in natural language processing. For example, the registration processing unit 111 first extracts a word or phrase representing a movement or state based on the result of the morpheme analysis as described above. In addition, the registration processing unit 111 may perform processing to sequentially extract noun phrases that become objects, adverb phrases that modify movements and states, adjective phrases, etc. For example, when morpheme analysis is performed in the speech recognition processing shown in the step S102 of the FIG. 4, the registration processing unit 111 may extract the part that needs interpretation based on the result of the speech recognition processing.

Alternatively, the storage unit 120 of the server system 100 may store in advance words representing movements, conditions, etc. that are highly necessary to be detected in the assistance. The registration processing unit 111 may extract the parts that need to be interpreted based on the comparison processing of those words and the text representing the starting condition. In addition, various modifications can be performed to extract the parts that need to be interpreted. For example, the parts that need to be interpreted may automatically be extracted using the learned model by perform machine learning for a neural network NN using the training data of both the know-how information and the part of the know-how information that needs to be interpreted.

In the step S202, the registration processing unit 111 identifies the device including the sensor required for processing based on the extraction result of the part that needs to be interpreted. For example, the registration processing unit 111 determines that it is necessary to detect the movement of the face (head) of the assisted person when determining the starting condition of “the face of the assisted person starts to wobble.” For example, the registration processing unit 111 identifies a camera capable of imaging the face of the assisted person, a wearable device that can be worn on the head and includes a motion sensor, etc., as a device required for processing. For example, the storage unit 120 of the server system 100 may store in advance one or more devices capable of detecting the movement of each body part of the assisted person. In addition, for example, the device required for processing may be automatically identified using the learned model by perform machine learning for a neural network NN using the training data of both the text information of the starting condition and the device required for processing.

Also, a camera or the like that can image the hand of the caregiver or the mouth of the assisted person is identified as a device required for processing to determine that the person “ate only a little meal from the spoon”.

In the step S203, the registration processing unit 111 determines whether the target user can use the specified device. The user here is a registered user who has registered the know-how information 121, for example, by performing the processing shown in the FIG. 4.

For example, for each user, the storage unit 120 stores in advance a device list of devices available to the user. The device list is information that identifies specific devices, such as smartphones, headsets, glass-type wearable devices, watch-type wearable devices, and devices that can obtain biometric information about the assisted person. More specifically, the device list may not only store information about a smartphone, but also the manufacturer of the smartphone, model number of the product, and so on.

Also, the devices available to the user are not limited to devices which the user wears and carries, the devices may be devices located in the nursing facilities, etc. For example, the device list may include cameras placed in the work environment of the target user, or may include other sensor devices. The sensors included in the sensor device can be modified in various ways, and various sensors can be used, such as temperature sensors, humidity sensors, illuminance sensors, barometric pressure sensors, activity meters, odor sensors, etc.

In the step S203, the registration processing unit 111 compares the device identified in the step S202 with the device list of devices available to the registered user. In the above example, the registration processing unit 111 determines whether each device included in the device list is capable of imaging the face of the assisted person or each device includes a motion sensor which is wearable on the head.

When the registration processing unit 111 determines that the specified device is not available by the registered user, the registration processing unit 111 omits the processing following the step S204. In this case, no device, etc. is associated with the registered know-how information 121. That is, the know-how information 121 is used in the form of text such as “if xxx is satisfied, do yyy” and the starting condition is not automatically determined. Note that the registration processing unit 111 instructs information representing the specified device is not available to the terminal device 200 via the communication unit 130, and the terminal device 200 may display a message.

When the registration processing unit 111 determined that the specified device is available by the registered user, the registration processing unit 111 determines the information necessary to automatically determine the starting condition. For example, the registration processing unit 111 determines a first processing algorithm for the device to collect device data, a second processing algorithm for performing processing on the collected device data, and parameters to be utilized in the second processing algorithm. Specifically, the device data is sensor information detected by sensors included in the device. For example, if the device including a camera such as a smartphone is specified, the device data (sensor information) is image data captured by the camera.

In the step S204, the registration processing unit 111 determines the first processing algorithm. In other words, the registration processing unit 111 determines the processing content when acquiring sensor information.

In the case of automating the determination of the starting condition, for example, that the face of the assisted person starts to wobble, the registration processing unit 111 needs to acquire, as the sensor information, information that causes a discernible difference between the case where the face starts to wobble and the case where the face does not start to wobble. That is, the sensor information in this case is information representing the result of detecting the movement of the face of the assisted person, and may be, for example, a one-second moving image of the area including the head of the assisted person or one-second time series data of a motion sensor mounted on the head of the assisted person.

For example, the storage unit 120 may store a table in which a plurality of first processing algorithms are associated with a word representing movement such as “wobble.” For an example of detecting whether or not the face starts to “wobble,” the first processing algorithm is an algorithm that causes the camera (imaging sensor) to take a moving image and output it in one-second increments. Alternatively, the first processing algorithm is an algorithm that causes the motion sensor to acquire acceleration data and angular velocity data in time series and output them in one-second increments. The registration processing unit 111 identifies the table based on the word extracted in the step S201 and performs processing to select one of the first processing algorithms included in the table. Although omitted in the FIG. 6, the registration processing unit 111 may perform processing to display a plurality of first processing algorithms included in the table in the terminal device 200 and may accept a selection operation by the user.

The device data (sensor information) can be collected by determining the first processing algorithm. Next, the processing unit 110 (the registration processing unit 111) performs processing to collect the multiple device data acquired by the devices and, for each of the multiple device data, performs processing to send a request to add a correct tag to the terminal device 200 used by the registered user. Here, the correct tag represents the result of determination by the registered user as to whether each of the multiple device data is satisfied with the starting condition. In this way, the information, which associates input data in the determination process of the starting condition with correct data to be output when this input data is input, can be collected. Based on these, the registration processing unit 111 can determine the parameters used in the second processing algorithm. The parameters will be discussed later. According to the method of this embodiment, since the determination criteria of the registered user is reflected in the parameters, the tacit knowledge of the registered user can be appropriately digitized. The specific processing is described below.

In the step S205, the registration processing unit 111 instructs the terminal device 200 to collect the device data as sample (Also described below as sample data) via the communication unit 130. For example, the registration processing unit 111 may send a program for performing the processing corresponding to the first processing algorithm described above. In the step S206, the terminal device 200 instructs the sensor to collect the sample data. Note that the sensor here may be included in the terminal device 200 or in a different sensor device from the terminal device 200. That is, in the step S206, the terminal device 200 may perform processing to control the internal sensor or may send information instructing external devices to collect data. The terminal device 200 or the sensor device starts collecting the sample data by installing the above program transmitted from the registration processing unit 111.

In the step S207, the sensor collects the sample data. In the step S208, the sensor transmits the collected sample data to the terminal device 200. In the step S209, the terminal device 200 sends the collected sample data to the server system 100. In the step S210, the registration processing unit 111 of the server system 100 stores the received sample data in the storage unit 120.

By the processing of the steps S207 to S210, one sample data is stored in the storage unit 120 of the server system 100. In this embodiment, the processing of the steps S207 to S210 is repeated until a predetermined number of sample data is accumulated. For example, after the registered users perform the processing shown in the FIG. 4, and then the sample data are gradually collected while continuing his or her normal work. For example, if the starting condition is registered, such as “If the face of the assisted person starts to wobble during a meal,” when the registered user performs meal assistance, the smartphone camera is turned ON to automatically collect the sample data that images the head of the assisted person. The registered users then continue their work, including the meal assistance, for a certain period of time to complete the collection of the predetermined number of sample data.

When it is determined that the collection of the prescribed number of sample data has been completed, in the step S211, the registration processing unit 111 performs processing to generate screen information for displaying the sample data. In the step S212, the registration processing unit 111 transmits the screen information to the terminal device 200. In the step S213, the display unit 240 of the terminal device 200 displays the sample data. The screen information here may be the display screen itself or information that can identify the display screen.

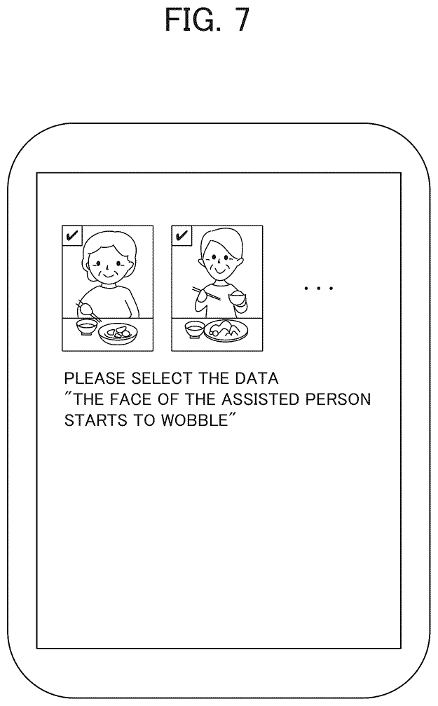

The FIG. 7 is an example of the screen displayed on the display unit 240 in the step S213. For example, when the sample data is a one-second moving image of the head of the assisted person, the display unit 240 displays a screen including thumbnails of each moving image. The display unit 240 may also display a screen prompting the user to input whether or not each sample data is satisfied with the starting condition. In the example of the FIG. 7, the display unit 240 displays the text “Please select the data ‘the face of the assisted person started to wobble.”

In the step S214, the terminal device 200 acquires correct data representing whether each sample data is satisfied with the starting condition. For example, the user performs an operation to select the data “the face of the assisted person starts to wobble” using the operation unit 250 based on the screen in the FIG. 7. For example, the terminal device 200 acquires the correct tag representing the correct answer as correct data corresponding to the selected sample data. In addition, the terminal device 200 acquires the incorrect tag representing an incorrect answer as the correct data corresponding to the sample data which is not selected at the completion of the user operation.

Also in the step S214, the terminal device 200 may obtain information regarding the point of view of the determination by the user. For example, when determining whether or not “the face of the assisted person starts to wobble,” the maximum amount of movement from the reference position may be used as a criterion. The reference position here may be, for example, the position of the face while sitting upright on a chair or a bed, or the center or the like in the captured image. Alternatively, it is possible to use the amount of movement of the head at one time as the criterion regardless of the reference position. That is, even if the same word “wobble” is extracted, the determination on the word may differ depending on the user.

For example, the storage unit 120 may store a table in which information representing multiple viewpoints is associated with a word representing movement, such as “wobble.” For example, a table stores two points of view: “the maximum movement angle of the head with respect to the reference position of the image is greater than the threshold alpha” and “one movement angle of the head is greater than the threshold beta.” The registration processing unit 111 may transmit a display screen prompting the selection of any of the multiple viewpoints included in the table, for example, in the step S212. Then, in the step S214, the terminal device 200 receives information about the viewpoint of determination together with acceptance of correct data.

In the step S215, the terminal device 200 transmits the correct data to the server system 100. When the viewpoint of determination is input as described above, the terminal device 200 transmits information about the viewpoint to the server system 100.

In the step S216, the registration processing unit 111 firstly receives the device data as an input and performs processing to determine a second processing algorithm for outputting output data representing whether or not the starting condition is satisfied. For example, the registration processing unit 111 determines the second processing algorithm based on the user input regarding the viewpoint of determination acquired in the step S215. For example, if the viewpoint is selected that the maximum movement angle of the head with respect to the reference position of the image is greater than the threshold alpha, the second processing algorithm indicates an algorithm that includes a process to determine the “maximum movement angle of the head relative to the reference position of the image” and a process of comparing the determined movement angle with the threshold alpha. If the viewpoint of “one head movement angle is greater than threshold beta” is selected, the second processing algorithm indicates an algorithm that includes a process of determining “one head movement angle” and a process of comparing the determined movement angle with threshold beta. Further, it is assumed that the specific processing content will differ between the case of moving images and the case of time-series acceleration data, etc. Therefore, the second processing algorithm may be determined according to the type of device (type of sensor) and the content of the first processing algorithm.

However, the parameters alpha and beta in the second processing algorithm are unknown. Therefore, in the step S216, the registration processing unit 111 performs processing to compute the parameters based on the sample data and the correct data. For example, the registration processing unit 111 obtains “the maximum movement angle of the head with respect to the reference position of the image” for the sample data according to the second processing algorithm. Then, the registration processing unit 111 performs processing to find the most probable alpha such that the movement angle becomes larger than the alpha for the sample data with the correct tag and the movement angle becomes the alpha or smaller for the sample data with the incorrect tag.

For example, the registration processing unit 111 may classify the sample data to which the correct tag is assigned and the sample data to which the incorrect tag is assigned using use SVM (support vector machine). For example, the registration processing unit 111 obtains a hyperplane separating the sample data to which the correct tag is assigned from the sample data to which the incorrect tag is assigned, and determines the parameters such as the alpha and the beta based on the hyperplane.

Note that the second processing algorithm is not limited to the above example and neural networks may be used. Hereafter, the neural networks are referred to as NN. For example, the storage unit 120 may store multiple NNs with different structures from each other as the multiple second processing algorithms. For example, the storage unit 120 stores a NN1, which is an NN suitable for processing with image data as input, and a NN2, which is a NN suitable for processing with velocity data and angular velocity data as inputs from the motion sensor. In the step S216, the registration processing unit 111 automatically or based on user input performs processing to select one of the multiple NNs including the NN1 and the NN2. Note that the NN1 is, for example, a CNN (Convolutional Neural Network), and the NN2 is, for example, a DNN (Deep Neural Network).

In the step S216, the registration processing unit 111 may perform learning processing using the NN. For example, the registration processing unit 111 obtains the output data by inputting the sample data into the NN and performing a forward operation using the weights at that time. Also, the registration processing unit 111 obtains an objective function (e.g., an error function such as a mean squared error function) based on the output data and the correct data, and updates the weight so that the error is reduced using an error back-propagation method or the like. The registration processing unit 111 may store the NN including the weights at the completing of learning in the storage unit 120 as a learned model. That is, when using the NN, the structure of the NN corresponds to the second processing algorithm and the weights correspond to the parameters.

In the step S217, the registration processing unit 111 stores the device used to acquire the sample data, the processing contents for the device data (sensor information) of the device, and the specified parameters in the storage unit 120 in association with the know-how information 121.

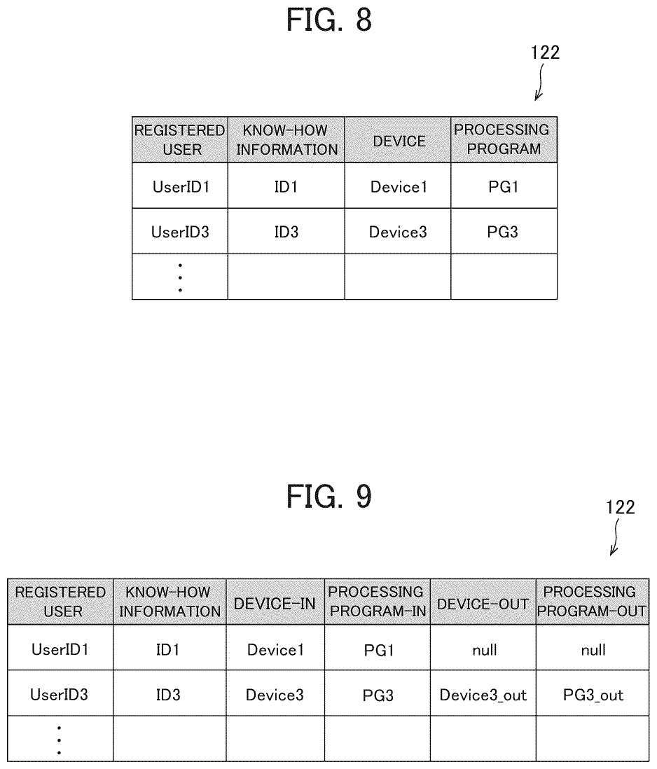

The FIG. 8 is an example of the registration information 122 stored in the step S217. As shown in the FIG. 8, the registration information 122 includes a user ID representing a user associated with a device, an ID representing know-how information 121, a device, and a processing program. The user here is, for example, a registered user who has registered the know-how information 121 using the FIG. 4. The devices included in the registration information 122 are, for example, information such as the manufacturer and model number of the device as described above. The processing program is, for example, a second processing algorithm and a set of parameters, and may be a NN including weights. In the following example, we assume that the processing program included in the registration information 122 is the second processing algorithm and parameter, but the processing program here may include the first processing algorithm described above. In this way, it is possible to manage the first processing algorithm for acquiring the device data to be processed by the second processing algorithm using the registration information 122.

By using the know-how information 121 in the FIG. 5 and the registration information 122 in the FIG. 8, it is possible to automatically determine the starting conditions. For example, with the know-how information 121 corresponding to ID 1 in the FIG. 5, whether the starting condition of “if 1” is satisfied or not can be automatically determined by performing processing according to PG1 for the device data of the Device 1.

As described above, the processing unit 110 (the registration processing unit 111) performs processing to identify the device used for the determination of the starting condition representing the condition information by performing the analysis processing for a text of the condition information (for example, the steps S202 and S203), and may associate the information representing the identified device with the know-how information 121 (for example, the step S217). In this way, since the condition information is associated with a specific device, it becomes possible to determine whether or not the starting condition is satisfied using the device.

It is possible to divide the know-how information 121 in this embodiment into 3 categories from the viewpoint of device association. Regarding to a first category, it is the know-how information 121 in which the device is determined to be usable in the step S203 and the processing in the step S217 is completed. This know-how information 121 can automatically determine the starting conditions because the second processing algorithm and the parameters are specified in addition to an association of the devices.

Regarding to a second category, it is the know-how information 121 in which the device was determined to be unusable in the step S203 and no processing was performed after the step S204. Since the know-how information 121 is used in the form of a text, whether the starting condition is satisfied or not is determined by the user himself or herself, for example.

Regarding to a third category, it is the know-how information 121 in which the device is determined to be usable in the step S203 but the processing in the step S217 is not completed. This know-how information 121 indicates that enough sample data to determine the parameters is not collected. For example, the registration processing unit 111 may not generate the registration information 122 for this know-how information 121, and may handle it in the same way as the know-how information 121 for which the device is determined to be unusable in the step S203. When enough sample data is accumulated in the future, the processing of the step S217 will be completed and registration information 122 will be generated, so that the starting condition can be determined automatically. In addition, there may be cases where the collection of sample data is not completed even after a lapse of time, and registration information 122 is not generated.

The FIG. 6 shows an example in which the parameters obtained in the step S216 are stored as they are in the step S217. However, the processing of this embodiment is not limited to this. For example, the registration processing unit 111 may use part of the combination of the sample data and the correct data as the validation data, and use the validation data to determine the correct rate of the determination processing using the second processing algorithm and parameters. The registration processing unit 111 transmits the correct rate to the terminal device 200. The terminal device 200 presents the correct rate and can accepts user input on whether or not to adopt the parameter. The registration processing unit 111 may perform the processing of the step S217 if the user inputs that the parameter is to be adopted. Also, if the user inputs that the parameter is not adopted, the registration processing unit 111 may reset the parameter, for example, and resume the collection of the sample data.

### 2.3 Determining the Correct Action

In the foregoing, we have described a method for automating the determination of the starting condition among the starting condition and assistance action included in the know-how Information 121. However, the method of this embodiment is not limited to this, and the processing related to the assistance action may be automated. For example, if there is know-how information 121 such as, “if the assisted person eats only a little meal from a spoon,” or “changing a posture that is easy to eat,” a process may be performed to obtain the correct answer for the “posture that is easy to eat,” or a process may be performed to warn the user if the posture that the user has made the assisted person take deviates from the correct one. It is possible to make the user perform the assistance action according to the registered know-how information 121 regardless of the skill level of the user by obtaining the correct assistance action.

The specific processing flow is the same as in the FIG. 6. That is, the registration processing unit 111 may extract the parts that need to be interpreted from the text representing the assistance action like the step S201. In the case of the above example, the registration processing unit 111 extracts the “posture that is easy to eat.”

Then, the registration processing unit 111 performs processing, which are same as the step S202 and S20, to identify devices including a camera for imaging the user and a motion sensor for detecting the posture as devices for detecting the posture that is easy to eat, and to determine whether the devices are available or not.

If the devices are available, as same as in the steps S207 to S215, the registration processing unit 111 collects the sample data representing the posture of the assisted person and instructs the user to add the tags for the collected results. For example, the sample data is a still image of the entire body of the user during a meal, and the registration processing unit 111 performs processing to accept a selection operation of a still image in an easy-to-eat posture among the multiple still images.

The registration processing unit 111, as same as in the step S216, determines the parameters based on the assigned tag. The second processing algorithm representing the processing content for the device data may be a comparison processing between the bending angle of the joints or the like and the threshold, or may be other processing. In addition, the NN may be used with the still image itself as an input. The parameter may be the above threshold or the weight of the NN.

As same as in the step S217, the registration processing unit 111 associates the registration information 122 including the device to determine the assistance action, the second processing algorithm, and the parameters with the know-how information 121.

The FIG. 9 is another example of registration information 122. As shown in the FIG. 9, the registration information 122 includes a user ID representing a user associated with the devices, an ID representing the know-how information 121, the device In representing a device used for determining the starting condition, and a processing program In representing processing program used for determining the starting condition, a device Out representing a device to be used for determining the assistance action, and a processing program Out, which is a processing program to be used for determining the assistance action. The device Out, like the device In, for example, is information such as the manufacturer and model number of the device. The processing program Out, like the processing program In, is for example the second processing algorithm and a set of the parameters, and may be a NN including weights.

For example, in the case of the know-how information 121 that “If the face of the assisted person starts to wobble during a meal”, “stop serving the meal”, it is easy to perform the assistance action of “stop serving the meal,” and there is less need to seek the correct action in the server system 100. Therefore, in this case, the processing described above for the assistance action may be omitted. For example, as in the know-how information 121 of ID 1 shown in the FIG. 9, the device Out and the processing program Out may set no data.

In addition, the second processing algorithm and parameters may not be determined due to factors such as insufficient sample data being collected, even though the device is determined to be usable for judging assisting behavior. Again, the device Out and the processing program Out become data free.

### 3. Use of Data

Through the above processing, the tacit knowledge of the user is accumulated as the know-how information 121. In addition, the registration information 122 for specifying a device or the like for automating the determination of the starting conditions and the determination of the assistance action is associated with respect to the know-how information 121 for which the conditions are satisfied. The techniques for using the acquired know-how information 121 are described below.

### 3.1 Search Processing

If a less skilled user can use the tacit knowledge of a skilled person, appropriate assistance can be performed regardless of the user's skill level. For example, each of the multiple users using the information processing system 10 selects one of the know-how information 121 stored in the storage unit 120 of the server system 100 and uses the selected know-how information 121.

The FIG. 10 is a diagram illustrating the process flow in which each user selects and uses the know-how information 121. First, in the step S301, the user uses the headset 300 to perform processing for inputting words to search. For example, the user utters the starting condition or the assistance action into the microphone of the headset 300.

In the step S302, the terminal device 200 performs the speech recognition processing and acquires text representing the starting condition or text representing the assistance action. In the step S303, the terminal device 200 transmits the acquired text as a search key to the server system 100.

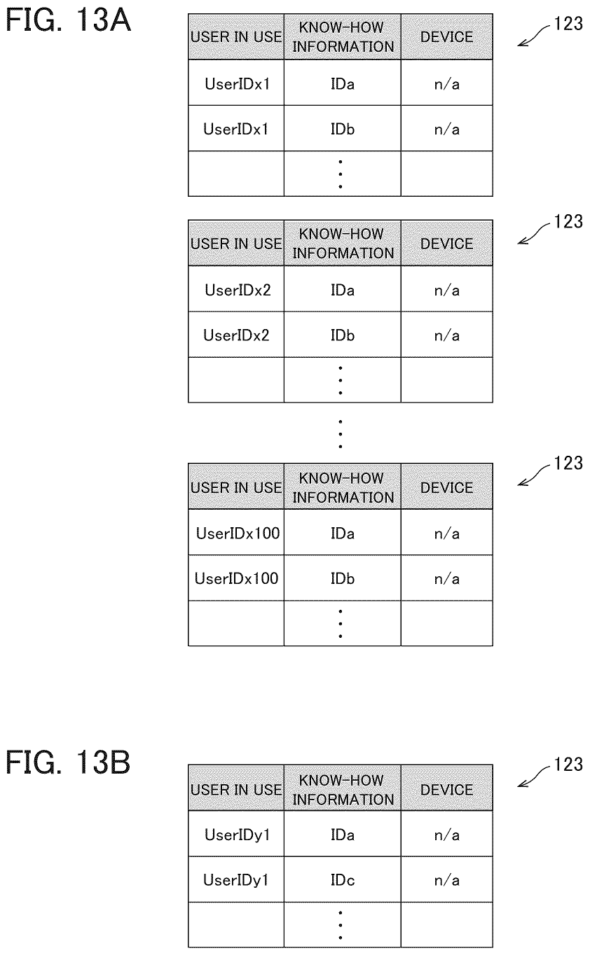

In the step S304, the server system 100 performs the searching processing using the acquired search key. That is, the processing unit 110 (the retrieval processing unit 112) of the server system 100 outputs any of the know-how information 121 among the plurality of know-how information 121 as a search result based on a search request that includes the search information (the search key) that is either a text corresponding to the starting condition or a text corresponding to the assistance action. For example, the retrieval processing unit 112 may output the know-how information 121 satisfying conditions such as the degree of matching with the search key as the search result. As described later with the reference to the FIG. 13A, the FIG. 13B, etc., the retrieval processing unit 112 may output as the search result the know-how information 121 in which the result of the first similarity determination processing satisfies a prescribed condition.

A case that the starting condition is used as the search key, for example, is a case that the user can not determine an appropriate assistance action. For example, suppose the user was able to recognize a situation in which the assisted person took a certain movement, the environment in which the assisted person lived changed in this way, etc., but did not know the assistance action to be performed in the situation. In this case, by performing search processing with the situation as the starting condition, the know-how information 121 representing the appropriate handling in the situation is provided.

And the assistance action represents a specific actions by the caregivers such as serving the meal with a spoon, talking to the assisted person, or changing the assisted person's posture etc. For example, although the user is aware of the assistance actions required to assist the assisted person in eating, excreting, etc., the user do not have enough skill to determine what kind of case the assistance action is performed, or a timing the assistance action is performed. In this case, by performing the searching processing based on the assistance action, the know-how information 121 representing the starting conditions for performing the assistance action is provided.

Thus, according to the method of the present embodiment, by performing the searching processing with the starting condition or the assistance action as the search key, it becomes possible to determine and present the know-how information 121 suitable for the user among the multiple know-how information 121 representing the tacit knowledge.

It should be noted that various modifications can be performed in the specific processing of the step S304. For example, when a text representing the starting condition is input as the search key, the retrieval processing unit 112 may determine that the know-how information 121 is satisfied with the condition if at least part of the text representing the starting condition included in the know-how information 121 matches the search key. In addition, when a text indicating the assistance action is input as the search key, the retrieval processing part 112 may determine that the know-how information 121 is satisfied with the condition if at least a part of the text indicating the assistance action included in the know-how information 121 matches the search key. Alternatively, the retrieval processing unit 112 may determine the similarity between the texts and determine that that the know-how information 121 is satisfied with the condition if the similarity is equal to or greater than the threshold.

In the step S305, the server system 100 transmits one or more pieces of the know-how information 121 determined to satisfy the condition to the terminal device 200. In the step S306, the display unit 240 of the terminal device 200 displays one or more acquired know-how information 121. In the step S307, the terminal device 200 accepts a selection operation by the user using, for example, the operation unit 250. That is, the user selects the know-how information 121 that the user wants to use from the know-how information 121 presented as the search result.

In the case that the registration information 122 shown in the FIG. 5 is associated with the know-how information 121, a specific device must be selected in order to fully use the know-how information 121. For example, suppose that the registered user who has registered the target know-how information 121 may use the smartphone which is a manufacturer AAA's model number BBB's smartphone. And the registration information 122 indicating the smartphone's information is stored. However, users using the know-how information 121 does not necessarily own a smartphone with the same manufacturer AAA model number BBB. Also, products of different model numbers from the same manufacturer may be used, or products of other manufacturers may be used, as long as the camera in the smartphone is used only. Furthermore, for example, when the registered user's smartphone is used for the purpose of imaging the face of the assisted person, a camera installed in a nursing bed or the like, a camera installed in a living room, a camera mounted on a movable device or the like may be used as the device used for the automatic determination. Therefore, when the know-how information 121 registered by a registered user is used by a user other than the registered user, a device selection operation may be performed for each user.

In the step S308, the terminal device 200 accepts a device selection operation by the user. For example, the storage unit 120 of the server system 100 stores the device list representing devices owned by the user who performed the searching processing, and may select and present a device close to the device included in the registration information 122 from the device list. For example, if the device of the registered user is a smartphone as described above, the retrieval processing unit 112 may perform processing to display a list of smartphones and similar devices owned by the user who performed the searching processing on the display unit 240 of the terminal device 200. If the registration information 122 is not associated with the know-how information 121, the processing of the step S308 is omitted.

Next, in the step S309, the terminal device 200 transmits the know-how information 121 selected by the user to the server system 100. In the step S310, the server system 100 updates the list information 123 representing the know-how information 121 in use by the target user. If the processing of the step S308 is performed, the information of the selected device is also transmitted and added to the list information 123.

As shown in the FIG. 10, the processing unit 110 can obtain requests for the use of any of the know-how information 121 stored in the storage unit 120 from multiple users. The storage unit 120 may associate and store the list information 123 including one or more the know-how information 121 in use for each of the multiple users. In this way, it becomes possible to appropriately manage the know-how information 121 used by each user among the large number of know-how information 121 accumulated in the storage unit 120.

For example, a less skilled user may increase the number of occasions in which the tacit knowledge of a skilled person can be used by actively using the know-how information 121. In addition, when the information is too much to grasp, it is possible to adjust the know-how information 121 to be used only for important information. In addition, the number of the know-how information 121 to be used may be reduced compared to the beginners, because the users with a certain level of experience can perform assistance appropriately without using the know-how information 121 in many cases.

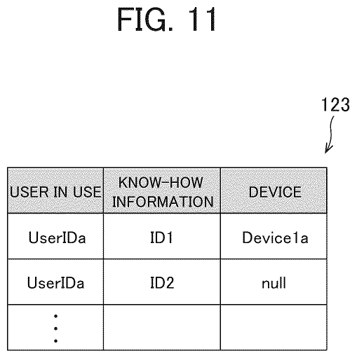

The FIG. 11 is an example of the list information 123. The list information 123 includes the information for identifying the user, the information for identifying the know-how information 121 being used by the user, and the information for identifying the device for using the know-how information 121. In the example of the FIG. 11, the user a corresponding to UserIDa is using the know-how information 121 of ID 1 registered by the registered user corresponding to User ID 1. As shown in the FIG. 8, the registered user had registered Device 1 in order to automate the know-how information 121 of ID 1. On the other hand, the user a selects Device 1a as the device for automating the know-how information 121 of ID 1, as shown in the FIG. 11. That is, even if the same know-how information 121 is used, the used devices may be different according to the user, and the storage unit 120 can store multiple devices associated with one of the know-how information 121.

Also, in the example of the FIG. 11, the user a is using the know-how information 121 of ID 2 registered by the registered user corresponding to UserID2. Since the registered user does not register any devices for the know-how information 121 of ID 2, any devices are not associated when the user a uses the know-how information 121. Note that the know-how information 121 with which no device is associated, such as ID 2 in the FIG. 11, is used, for example, in the form of text. For example, if the information such as “meal” is included in the know-how information 121 as additional information, the text corresponding to the know-how information 121 may be notified to the user at the time of starting the meal assistance.

On the other hand, the know-how information 121 with which devices are associated, such as ID 1 in the FIG. 11, enables automation such as the determination of starting conditions.

The FIG. 12 is a diagram illustrating the processing flow using the know-how information 121 with which devices are associated. First, if the know-how information 121 is added to the list information 123, the corresponding device starts collecting the sensor information in the step S401. Note that the sensor information may be collected at any time or used in a specific situation related to the know-how information 121. For example, if the know-how information 121 includes the information such as “excretion” as the additional information, the device may start collecting the sensor information at the timing when the excretion assistance is started.

In the step S402, the sensor transmits the sensor information to the terminal device 200. If the device here is the terminal device 200, the processing in the step S402 corresponds to the transfer of data from the sensor to the processor in the terminal device 200. In the step S403, the terminal device 200 transmits the sensor information to the server system 100.

In the step S404, the processing unit 110 automatically determines the starting condition based on the sensor information. For example, the processing unit 110 determines the second processing algorithm and parameters based on the registration information 122 in the FIG. 8. The processing unit 110 obtains output data indicating whether or not the starting condition is satisfied by performing processing according to the above second processing algorithm and parameters using the sensor information as the input data.

If the processing unit 110 determines that the starting condition is satisfied, in the step S405, the processing unit 110 identifies the assistance action based on the know-how information 121. In the step S406, the processing unit 110 transmits the information representing the specified assistance action to the terminal device 200. In the step S407, the terminal device 200 transmits the information representing the assistance action to the headset 300. In the step S408, the headset 300 uses a speaker to announce the assistance action. Here, the information representing the assistance action is text, and the processing in the step S408 may be a speech reading processing. However, as described above with the reference to the FIG. 9, the processing unit 110 may obtain the correct answer of the assistance action and perform notification based on the correct answer.

As described above, according to the method of the present embodiment, it is possible to convert the tacit knowledge of the skilled user into data and to make the less skilled user provide appropriate assistance. For example, a less-skilled user can provide assistance equivalent to that of a skilled person, thus improving the reproducibility of assistance. In addition, the variation in care skills is suppressed, and organizational management is facilitated, thereby reducing the occurrence of incidents such as falling of the assisted person. As a result, for example, in the nursing facilities, the occurrence of vacancies due to hospitalization and the occurrence of overtime due to the preparation of accident reports can be reduced. Curbing incidents also curbs the users from becoming too risk-sensitive, which can reduce stress and consequently turnover. In addition, by improving the skills of the users and the working environment, it is possible to improve the satisfaction of the assisted person and his or her family and to improve the quality of life (QOL) of the assisted person and his or her family.

The information processing system 10, the server system 100, the terminal device 200, etc., of this embodiment may realize part or most of its processing by a program. In this case, a processor such as a CPU performs a program to realize the information processing system 10 or the like of this embodiment. Specifically, a program stored in a non-transitory information storage medium is read, and a processor such as a CPU performs the read program. Here, an information storage medium (a medium that can be read by a computer) stores programs, data, etc., and its function can be realized by an optical disk, an HDD, or a memory (card-type memory, ROM, etc.). Then, a processor such as a CPU performs various processing of this embodiment based on a program stored in an information storage medium. That is, a program for making the computer function as a part of this embodiment is stored in the information storage medium.

In addition, the method of the present embodiment includes a step for receiving a registration request of the know-how information 121 including the information which associates condition information representing a starting condition and assistance information representing assistance action to be performed if the condition information is satisfied and a step for outputting one of a plurality of the know-how information 121 stored by a plurality of the registration requests as the search result based on the search request including information to identify either one of the starting condition and the assistance actions.

### 3.2 Determination of Similarity Between the Know-How Information

In addition, the server system 100 (the similarity determination unit 113) of this embodiment may perform processing for determining the similarity between a certain know-how information 121 and other know-how information 121. For example, the retrieval processing unit 112 may determine in the step S304 of the FIG. 10 that the conditions are satisfied not only for the know-how information 121 extracted using the search key but also for similar know-how information 121 which is similar to the certain know-how information 121.

For example, the similarity determination unit 113 may determine the similarity of the two pieces of the know-how information 121 based on the additional information included in the know-how information 121. For example, as shown in the FIG. 5, the additional information includes the type of assistance and words representing the attributes of the assisted person. When the same word is included in the two pieces of the know-how information 121, the similarity determination unit 113 determines that the similarity is high. Alternatively, a synonym is defined for each word, and the similarity determination unit 113 may determine that the similarity of the two pieces of the know-how information 121 is high if the synonym of the word included in one of the know-how information 121 is included in the other know-how information 121.

Alternatively, the similarity determination unit 113 may determine the similarity based on text mining. For example, the similarity determination unit 113 performs processing of the text mining for at least one of the texts representing the starting condition and the assistance action. For each of the words extracted by the text mining, the similarity determination unit 113 obtains the tf-idf representing the importance of the word, where tf is the frequency of appearance of the word, and idf is the frequency of reverse documents, where tf-idf is an index that increases the importance of the word with the high frequency of appearance and decreases the importance of the word appearing in many documents. For example, the similarity determination unit 113 obtains a vector that associates tf-idf as a value with each word appearing in the know-how information 121. The similarity determination unit 113 obtains a vector for each of the two pieces of the know-how information 121, and determines a degree of the similarity of the two pieces of the know-how information 121 based on the angle Theta formed by the obtained two vectors. For example, the degree of the similarity is cos Theta. However, various methods for determining the similarity of two documents are known, and they are widely applicable in the present embodiment. Also, the portion subject to text mining is not limited to at least one of the starting condition and the assistance action, and may include the additional information.

Also, the similarity determination unit 113 may obtain the similar know-how information 121 for the certain know-how information from the viewpoint of whether the know-how information is often used with the certain know-how information 121.

For example, the multiple know-how information 121 includes the first know-how information, the second know-how information and the third know-how information, and the processing unit 110 (the similarity determination unit 113) performs similarity determination processing to determine the similarity between any two pieces of the multiple know-how information 121 stored in the storage unit 120. At this time, the similarity determination unit 113 determines the similarity based on the number of users whose list information 123 includes the first know-how information and the second know-how information, and the number of users whose list information 123 includes the first know-how information and the third know-how information.

The FIGS. 13A and 13B illustrate the similarity determination processing. The FIG. 13A is an example of the list information 123 about users who are using both the first know-how information corresponding to IDa and the second know-how information corresponding to IDb. In the example in the FIG. 13A, 100 people corresponding to UserIDx1 to UserIDx100 are using both the first and second know-how information.

The FIG. 13B is an example of the list information 123 about users who are using both the first know-how information corresponding to IDa and the third know-how information corresponding to IDc. In the example of the FIG. 13B, only one user corresponding to UserIDy1 is using both the first know-how information and the third know-how information.

In this case, the second know-how information is easily used with the first know-how information, and the third know-how information is not easily used with the first know-how information. The similarity determination unit 113 determines that the similarity between the first know-how information and the second know-how information is relatively higher than the similarity between the first know-how information and the third know-how information.

it is possible to present the know-how information 121 which is useful to use with the certain know-how information to users as the similar know-how information. For example, it is possible to present a combination of useful know-how information 121 to a user who has performed the above the search processing using the FIG. 10. The first know-how information and the second know-how information here may differ in the type of assistance. For example, if the first know-how information is related to the meal assistance, the second know-how information is related to the excretion assistance and the transferring or moving assistance. In this way, the know-how information 121 that is determined to have low similarity from the viewpoint of the additional information can be included in the similar know-how information.

### 3.3 Similar Determination in Registration Processing

In addition, an example of using similarity determination processing between the know-how information 121 in the search processing has been described above. However, the cases using the result of similarity determination processing is not limited to this.

For example, if the registered user newly registers the know-how information 121, the similar know-how information similar to the know-how information 121 to be registered may be presented to the registered user. This processing may be performed after the step S103 in the FIG. 4, alternatively may be performed after the step S216 in the FIG. 6. For example, if the correct rate is presented after the operation of the parameters in step S216 or if the user's selection regarding the adoption or non-adoption of the parameters is received after the operation of the parameters in step S216, the similar know-how information may be presented together with receiving the selection operations.

If the registered user registers the know-how information 121, the know-how information 121 is information representing the tacit knowledge of the registered users. Therefore, the similar know-how information similar to this know-how information 121 is highly likely to be useful information for the registered users. Therefore, by presenting the similar know-how information at the time of registration of the know-how information 121, it becomes possible to efficiently use the tacit knowledge.

The similarity determination processing here is performed based on the additional information as described above, based on the text mining, or based on the number of users using two pieces of the know-how information 121 together. However, in this case, the know-how information 121, which is text, may have already been registered (the step S104), but the association of devices, etc. (the step S217) has not been completed. Therefore, the use of know-how information 121 by other users may not be progressing. Therefore, the similarity determination unit 113 may omit a determination based on the number of users using the two pieces of the know-how information 121 together in the similarity determination processing, and various variations can be performed in the specific similarity determination processing.

### 3.4 Recommended Users and Recommended Facilities

The processing unit 110 (the similarity determination unit 113) may also perform second similarity determination processing to determine the similarity of the list information 123 corresponding to the first user among the multiple users and the list information 123 corresponding to the second user different from the first user.

As described above, the list information 123 of the first user is a set of the know-how information 121 being used by the first user, and the list information 123 of the second user is a set of the know-how information 121 being used by the second user. That is, if the similarity between the list information 123 can be determined, the users who are using the same tacit knowledge can be identified.

For example, suppose a user has experience of assisting a prescribed assisted person, and his or her assistance is evaluated. The quality of assistance may be evaluated by the assisted person by himself or herself, by a family member of the assisted person, or by a care manager. In addition, the quality of the assistance may be evaluated based on whether the assisted person smiles a lot or not, based on the results of face recognition processing of the face image of the assisted person.

The user whose assistance content is being evaluated may be, for example, a home caregivers who provide nursing care at home or a family member of the assisted person. In this case, from the perspective of appropriately assisting the assisted person, it is desirable for the user to continuously provide assistance. However, there may be situations where the user is unable to provide assistance due to factors such as family members having some errands to attend, or home caregivers taking time off, being transferred or changing their jobs. In addition, even if the user is able to assist, it may be necessary to look for different home caregivers from the perspective of cost.

In this case, the processing unit 110 (the similarity determination unit 113) performs the second similarity determination processing based on the list information 123 corresponding to the user who has experience of assisting the assisted person and the list information 123 of the multiple users, and performs processing to determine from the multiple users the recommended user recommended for assisting the assisted person based on the result of the second similarity determination processing.

The FIG. 14 is a diagram illustrating the process flow for determining the recommended user. First, the family member or the care manager of the assisted person performs a search request operation for the recommended user. For example, the family member or the like uses the terminal device 200 to enter a user ID specifying the user who performed the desired assistance for the assisted person. For example, if the user who performs the search request operation is the family member of the assisted person, enter his or her own user ID if the family member performs daily assistance by himself or herself, or enter the user ID of the home caregiver if the family member requests daily assistance from the home caregiver. In the step S501, the terminal device 200 accepts the search request operation.

In the step S502, the terminal device 200 transmits a recommended user search request including the user ID to the server system 100. In the step S503, the similarity determination unit 113 of the server system 100 identifies the list information 123 of the users who performed the desired assistance based on the user ID transmitted from the terminal device 200. In the step S504, the similarity determination unit 113 performs the second similarity determination processing to determine the similarity between the list information 123 identified in the step S503 and the list information 123 of other users.

The FIG. 15 is a flowchart explaining the second similarity determination processing in the step S504. In the step S601, the similarity determination unit 113 extracts one of the know-how information 121 included in one of the list information 123. In the step S602, the similarity determination unit 113 performs the first similarity determination processing to determine the similarity between the know-how information 121 extracted in the step S601 and each of one or more the know-how information 121 included in other list information 123. In the step S603, the similarity determination unit 113 stores the maximum value of the calculated similarity among the multiple similarities as a score of the extracted know-how information 121.

In the step S604, it is determined whether the calculation of scores has been completed for all the know-how information 121 included in one of the list information 123. If the score calculation has not been completed, the similarity determination unit 113 returns to the step S601, extracts the other know-how information 121, and performs the first similarity determination processing and stores the scores for the extracted know-how information 121.

If the calculation of scores has been completed for all the know-how information 121, in the step S605, the similarity determination unit 113 obtains the second similarity which is the result of the second similarity determination processing based on the calculated score or scores. The second similarity here may be the sum of one or more scores, an average, or other information such as the result of weighted addition. The FIG. 15 shows an example of the second similarity determination processing, and the specific processing is not limited to this, and various modifications can be performed.

For example, if the type of assistance to be used is known in advance, the similarity determination unit 113 may perform processing limited to that type of assistance. For example, it is assumed that the similarity determination unit 113 accepted a search request for a recommended user to provide the meal assistance of the assisted person in the step S502. In this case, the similarity determination unit 113 may extract the know-how information 121 with which “the meal” is associated as additional information from each of the two list information 123 to be compared and perform the second similarity determination processing for the extracted results. Alternatively, the similarity determination unit 113 may determine the second similarity based on comparison processing between the distribution information representing the distribution of the know-how information 121 contained in one list information 123 and the distribution information of the other list information 123.

Using the process shown in the FIG. 15, the second similarity determination processing may be completed between the list information 123 of the users who performed the desired assistance and the list information 123 of one of the multiple users. The similarity determination unit 113 performs the same processing as in the FIG. 15 for the other users.

In the step S505, the similarity determination unit 113 identifies the user corresponding to the list information 123 with the maximum second similarity as the recommended user. In the step S506, the server system 100 sends information about the recommended user to the terminal device 200. In the step S507, the display unit 240 of the terminal device 200 displays information about the recommended user.

In the step S505, the similarity determination unit 113 identifies the user corresponding to the list information 123 whose second similarity is equal to or greater than a prescribed threshold as the recommended user. The recommended users in this case are not limited to one person. In the step S506, the server system 100 sends information about one or more recommended users to the terminal device 200. In the step S507, the display unit 240 of the terminal device 200 displays information about the recommended user.

In this way, it will be possible to present to the family of the assisted person, the care manager, etc., as a recommended user the information of the user who has a high probability of providing the same assistance as the user who provided appropriate assistance in the past. Therefore, by requesting the recommended user to provide assistance, it becomes possible to increase the satisfaction of the assisted person and his or her family.

Also, for each of the multiple facilities, the storage unit 120 may store the facility list information 124 which is a collection of the know-how information 121 registered by one or multiple users who belong to each facility. The facility here represents the organization to which the user who assists the assisted person belongs and may be a nursing facility, hospital or other facility.

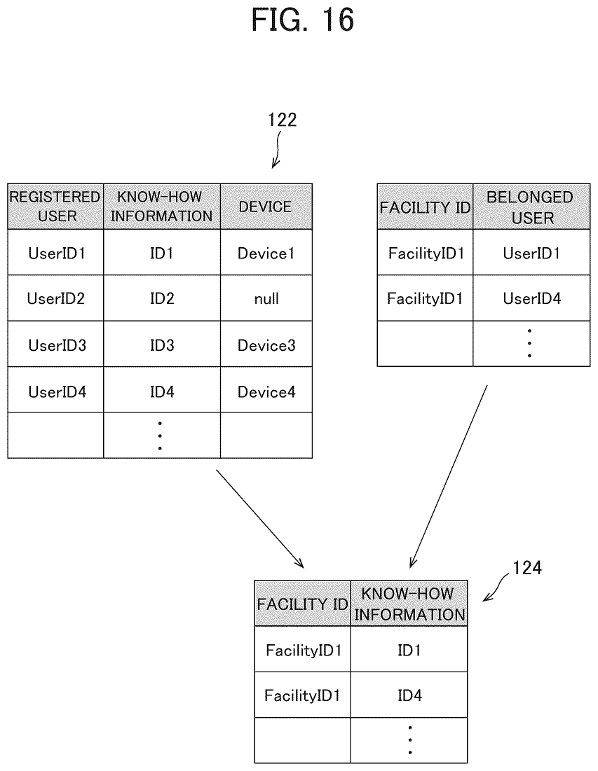

The FIG. 16 illustrates the processing for obtaining the facility list information 124. As shown in the FIG. 16, the storage unit 120 stores the registration information 122. The details of the registration information 122 are as described above using the FIG. 8, and the registration information 122 includes a registered user who registers the know-how information 121 and an ID that identifies the know-how information 121. The storage unit 120 may also store information associating the facility with a user belonging to the facility. In the example shown in the FIG. 16, the user corresponding to UserID1 and the user corresponding to UserID4 belong to the facility corresponding to FacilityID1.

The processing unit 110 requests the facility list information 124 based on two pieces of information. In the example of the FIG. 16, the user of UserID1 has registered the know-how information 121 of ID 1, and the user of UserID4 has registered the know-how information 121 of ID 4. Therefore, the facility list information 124 of the facility of Facility ID 1 includes the know-how information 121 of ID 1 and ID 4.

If the user who belongs to the facility registers the know-how information 121, there is a high probability that the relevant know-how information 121 corresponds to tacit knowledge acquired while working at the target facility. That is, the facility list information 124 can be said to represent tacit knowledge unique to the facility.

The processing unit 110 (the similarity determination unit 113) may perform a third similarity determination processing to determine the similarity between the list information 123 corresponding to a prescribed user and the facility list information 124, and determine a recommended facility recommended for the prescribed user or a recommended user recommended for the prescribed facility based on the result of the third similarity determination processing. The specific flow of the third similarity determination processing is the same as the second similarity determination processing shown in the FIG. 15. That is, the third similarity determination processing is performed on the basis of the similarity between the know-how information 121 included in the facility list information 124 and the know-how information included in the list information 123, and as a result, the third similarity degree is obtained. Various modifications can also be made to the third similarity determination processing, such as comparing the distribution information of the list information 123 with the distribution information of the facility list information 124.

For example, the similarity determination unit 113 performs the third similarity determination processing based on the list information 123 corresponding to the user who has experience of assisting the assisted person and the facility list information 124 of the multiple facilities, and determines the recommended facility recommended for assisting the assisted person from the multiple facilities based on the result of the third similarity determination processing. In this way, it is possible to recommend a facility that can perform assistance for the target assisted person, thereby improving the satisfaction of the assisted person and the family and improving the work efficiency of the care manager. As a result, early leaving from the facilities can be curbed.

Alternatively the third degree of similarity determination processing may be used by users such as home caregivers, caregivers, nurses, physical therapists, occupational therapists, speech pathologists, etc. For example, if these users starts looking for jobs, it is desirable to select a facility with a matching assistance policy. The users have the advantage of being able to make use of the assistance experience they have accumulated. The facilities can also reduce the educational burden and reduce turnover if they can hire users who fit their policies. According to this method, it is possible to match the users with the facilities based on the third similarity determination processing based on the list information 123 and the facility list information 124.

The third similarity determination processing may be triggered by users such as caregivers. For example, a user who is considering changing jobs transmits a search request for recommended facilities to the server system 100 using the terminal device 200. The server system 100 performs the third similarity determination processing to determine the similarity between the target user's list information 123 and the multiple facility list information 124. The server system 100 transmits the information of the facility determined to have a high degree of the third similarity to the terminal device 200, and the display unit 240 of the terminal device 200 displays the information of the facility. For example, the display unit 240 may display a list of facilities for which the third similarity is determined to be greater than or equal to the threshold, or may display a list of a predetermined number of facilities in order of the third similarity being higher. Also, the facilities that satisfy the conditions of the third similarity and are at a distance from the user's residence of less than or equal to a predetermined threshold may be displayed with the map.

Alternatively, the third similarity determination processing may be performed by a user, such as a facility manager, as a trigger. For example, a person in charge of a facility recruiting personnel uses the terminal device 200 to transmit a search request for a recommended user to the server system 100. The server system 100 performs the third similarity determination processing to determine the similarity between the facility list information 124 of the target facility and the multiple user list information 123. The server system 100 transmits the information of the user determined to have a high degree of the third similarity to the terminal device 200, and the display unit 240 of the terminal device 200 displays the information of the user. For example, the display unit 240 may display a list of users whose third similarity is determined to be equal to or greater than the threshold, or display a list of a predetermined number of users from the order of the third similarity to the highest. In addition to the third similarity condition, a filtering processing based on information such as the user's residence may be performed.

The FIG. 17 is an example of a user page in which information about a prescribed user is displayed in the service usage screen of the information processing system 10 of this embodiment. As shown in the FIG. 17, the user page may include an area RE 1 displaying the know-how information 121 in use by the target user, an area RE 2 displaying the registered know-how information 121, an area RE 3 displaying the know-how information 121 recommended for use, and an area RE 4 displaying recommended facilities.

The know-how information 121 included in the list information 123 described above is displayed in the area RE 1 using, for example, the FIG. 11. The know-how information 121 included in the registration information 122 described above is displayed using, for example, the FIG. 8. The know-how information 121 similar to the know-how information 121 in use or the registered know-how information 121 is displayed in the area RE 3. The know-how information displayed in the area RE 3 is obtained based on, for example, the first similarity determination processing described above. The information about facilities determined to have high third similarity by the third similarity determination processing is displayed in the area RE 4.

By making such a display, it is possible not only to register the know-how information 121 and easily grasp the usage situation but also to present the know-how information 121 that is unused but has been determined to be suitable for the user , and to present facilities where the assistance policy is close to the user. That is, by using the screen shown in the FIG. 17, useful information for the user can be presented in an easy-to-understand manner.

As shown in the FIG. 17, the area shown in RE 1 may display the device associated with the know-how information 121 in addition to the know-how information 121 in use. The devices displayed here are the devices included in the list information 123 as shown in the FIG. 8 or the FIG. 9. In this way, the information regarding to the devices used in the know-how information 121 in use can be presented to the user in an easy-to-understand manner.

Also, as shown in the FIG. 17, in the area shown in RE 2, information representing the registration status of the device regarding the know-how information 121 registered by the target user may be displayed. For example, regarding to the know-how information 121 with which the device in the FIG. 6 is not associated (No in the step S203), an object including the text “No device” indicating that the device is not associated is displayed.

Also, in the case that although the device was associated (Yes in the step S203), but the know-how information 121 is not ready for the user to add the correct data because not enough sample data have been collected (the loop of the steps S207 to S210 is continuing), an object including the text “ Not ready ” indicating that enough sample data have not been collected is displayed.

Also, in the case that because enough sample data have been collected (complete loop of the steps S207 to S210), the know-how information 121 is ready for the user to add the correct data, an object including the text “ready” indicating that enough sample data have been collected is displayed. For example, if the user performs a selection operation on an object displayed as “ready,” the processing from the step S211 in the FIG. 6 and onward is started.

Also, not shown in the FIG. 17, in the case of the know-how information 121 that correct data addition by the user, determination of the second processing algorithm and parameters (the step S216), and generation of the registration information 122 (the step S217) have been completed, an object including the text “completed” indicating completion of setting as to the know-how information is displayed. As described above, by displaying not only the information identifying the know-how information 121 but also the information associated with the know-how information 121, it becomes possible to present the usage situation and registration situation of the know-how information 121 of the target user in an easy-to-understand manner.

### 3.5 HACs (Hospital-Acquired Conditions)

In the hospital admissions, the hospital-acquired conditions (HACs) is known. The HACs refers to the occurrence of a disease after admission that is different from the original intended treatment. The HACs are inherently preventable, indicates the occurrence of the deficiencies in patient management. In the United States, for example, the hospitals are required to pay for the medical care of the HACs, and controlling the HACs is very important.

The HACs include events such as foreign body residue after surgery, air embolism, blood inconsistency, pressure ulcers, falls, trauma, fractures, dislocations, injuries in the skull, catastrophic injuries, burns, other injury injuries, inadequate blood glucose control, urinary tract infections caused by catheters, catheter-related infections, surgical site infections/mediastinitis after cardiac bypass surgery, surgical site infections after bariatric surgery, laparoscopic gastric bypass, gastric pouch augmentation, limited laparoscopic gastric surgery, surgical site infections after orthopedic surgery, cardiac implantable electronic device surgical site infections, deep vein thrombosis/pulmonary embolism after orthopedic surgery, total knee arthroplasty, hip replacement, iatrogenic thoracic pneumothorax, etc. The above are examples of the HACs, and various modifications can be made to specific events.

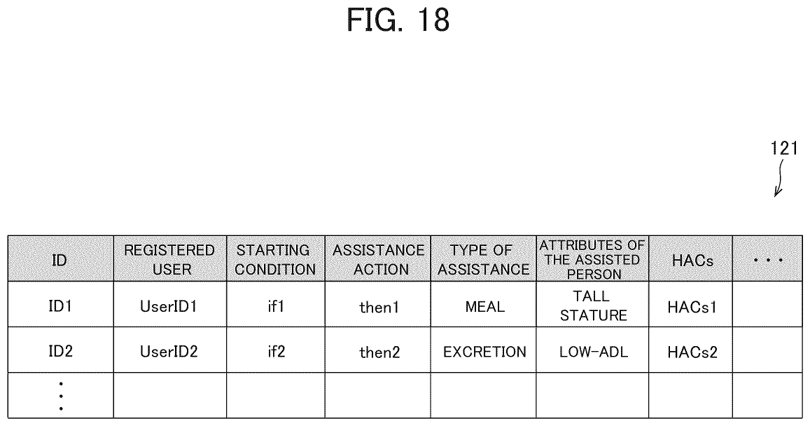

The know-how information 121 in this embodiment may include information representing the hospital-acquired conditions. The FIG. 18 is an example of the know-how information 121. The HACs1 and the HACs2 shown in the FIG. 18 each represent one of the above events. As shown in the FIG. 18, by associating the HACs with the know-how information 121, it becomes possible to identify whether each know-how information 121 is effective to suppress for which type of HACs. The information indicating the HACs may be obtained by performing a series of utterances such as “To suppress the HACs1, if do xxx, do yyy.” Alternatively, the server system 100 may ask a question such as “What kind of the HACs suppression does it help?” and the information representing HACs may be obtained as an answer to the question.

For example, the person who actually assist patients in the hospitals is the Certified Nursing Assistants (CNAs). Therefore, the CNAs add the necessary know-how information 121 to the list information 123 and uses it in a specific assistance situation, as described above using the FIG. 10. At that time, the retrieval processing unit 112 may decide the know-how information 121 to be presented to the target CNAs based on the comparison processing between the HACs generated in the past by the CNAs itself and the information representing the HACs including in the know-how information 121. For example, appropriate suppression of the HACs can be achieved by having the CNAs who causes the pressure ulcers use know-how information 121 that is considered to be effective for pressure ulcer suppression.

However, if the selection and use of the know-how Information 121 on HACs are completely entrusted to the CNAs, The know-how Information 121 may not be used properly. For example, even if a certain CNA causes the pressure ulcers for the assisted person, the CNA may not be aware of its importance, or may not be able to afford to update the list information 123 due to its heavy daily workload. The HACs, however, are not limited to the CNA individual matters, but are relevant to the evaluation of the hospital to which the CNA belongs. It is also known that the medical costs associated with HACs are enormous, which may affect the management of the hospitals in countries that have adopted a system in which the hospitals pay costs for the HACs as described above.

Therefore, in this embodiment, when managing the list information 123 of the users who provide the direct assistance, the operation may be enabled not only by the user himself or herself but also by a management user who directs and supervises the user. For example, the multiple users in this embodiment include a working user who is directed by the management user. The working user is a person who directly assists the assisted person, for example, the CNAs described above.

In addition, the management user is a person who directs and supervises the working user, such as a registered nurse (RN; a Registered Nurse). However, the management user and the working user only need to be in a hierarchical relationship, and their specific positions are not limited to the RN and the CNAs. The method of this embodiment can also be extended to an organization with three or more levels of command and control.

The management user is a user who can use the information processing system according to this embodiment and has the authority to view information related to the working user (for example, the user page of the working user as shown in the FIG. 17). The management user may be a user who uses the know-how information 121 in the assistance by generating his or her own list information 123 in the same manner as the user described above. The management user may be a user who mainly manages the working user and does not provide actual assistance.

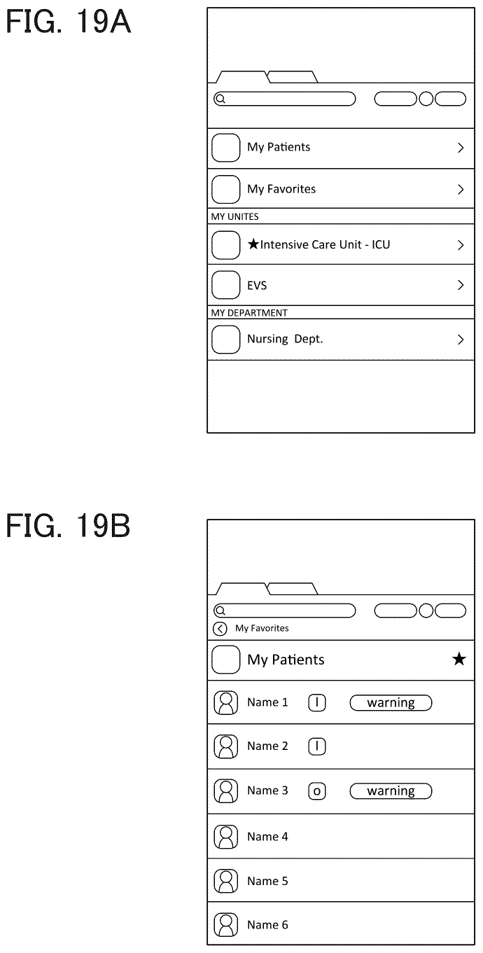

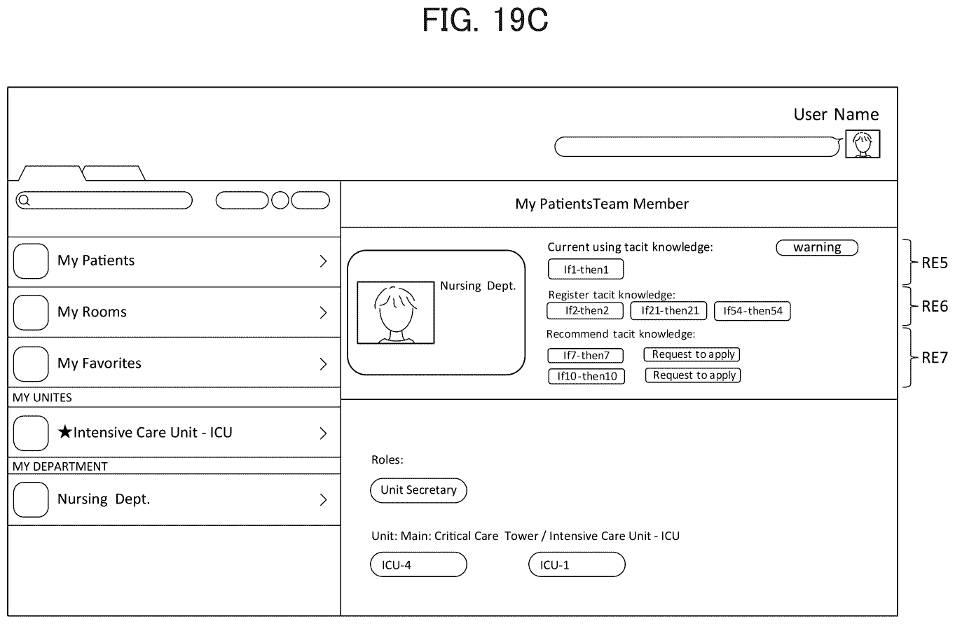

The FIGS. 19A to 19C are examples of the user pages that display information related to a prescribed management user and are the service usage screens of the information processing system 10 of this embodiment. As shown in the FIG. 19A, the management user's user page displays objects used for transitioning to information related to the patient in charge, objects used for transitioning to information registered as his or her favorite, objects used for transitioning to information related to the organization to which he or she belongs, etc. When the selection operation of each object is performed, the transition to the display screen of the corresponding information is performed.

The FIG. 19B is an example of the screen displayed when, for example, an object including the text “My Patients” in the FIG. 19A is selected. In the example of the FIG. 19B, the target management user RN, directs and supervises the multiple CNAs. For example, The Name 1 through Name 6 represent the full name of the CNAs. Each CNA is responsible for assisting one or more patients. That is, the information related to the patient in charge may be information about multiple working users who are under the supervision of the management user and are directly responsible for assisting the patient. By displaying such an indication, it is possible to present the managed working users to the management users in an easy-to-understand manner.

The processing unit 110 may decide recommended know-how information to be used by the working user from a plurality of the know-how information 121 on the basis of the occurrence status of hospital-acquired conditions of the working user, and may present the decided recommended know-how information to the management user. For example, a prescribed CNA has a history of causing pressure ulcers within a prescribed time period. The prescribed period here is, for example, the period from now to six months ago, but the specific period allows various modifications to be performed. In this case, in order to suppress the occurrence of the HACs again by the CNA, it is advisable to have the CNA use the know-how information 121 associated with the pressure ulcers.

Therefore, the processing unit 110 can determine recommended know-how information based on, for example, the HACs with a history of occurrence and the information about HACs included in the know-how information 121. For example, the processing unit 110 may display a warning if there is a working user who has a history of occurrence of HACs but has not used the corresponding know-how information 121. In the example of the FIG. 19B, an object including the text “Warning” is displayed in the area corresponding to the CNA corresponding to Name 1.

The FIG. 19C is an example of the screen displayed when the selection operation of a prescribed user is performed, for example, in the FIG. 19B. As shown in the FIG. 19C, when the prescribed user is selected, the face shot, name, affiliation, etc. of the selected CNA are displayed, and the know-how information related to the CNA is also displayed. Specifically, the screen in the FIG. 19C includes an area RE 5 that displays the know-how information 121 in use by the target CNA and an area RE 6 that displays the registered know-how information 121. Also, if the user for whom the warning display was made is selected, the screen in the FIG. 19C may include area RE 7 for displaying the recommended know-how information. For example, “if 7—then 7” and “if 10—then 10” displayed in the area RE 7 of the FIG. 19C represent the recommended know-how information.

Thus, it is possible for the user who is in a position to manage one or more working users to appropriately grasp the occurrence status of HACs and the usage status of the know-how information 121 of each working user. In addition, if the know-how information 121 for suppressing the HACs is not fully utilized, it becomes possible to inform the management user about it.

If the processing unit 110 receives a request from the management user to add the recommended know-how information, it may also perform processing to add the recommended know-how information to the list information 123 of the working user. More specifically, if the processing unit 110 receives a request from the management user to add the recommended know-how information, the processing unit 110 may perform processing to add the recommended know-how information to the list information 123 of the working user without permission from the working user. In this way, it is possible for the management user to manage the list information 123 of the working users. Thus, even if the working user for some reason did not use the appropriate know-how information 121 for HACs suppression, it is possible for the management user to correct it. This allows the hospital as a whole to promote the suppression of HACs, etc., because it can avoid being left to individual working users. Furthermore, by controlling occurrence of the HACs, it is possible to control health care costs and the incidence of patients throughout society.

For example, as shown in the RE 7 of the FIG. 19C, an object including the text “Request to apply” may be displayed in the area corresponding to each of the recommended know-how information. The object is used by the management user to request the additional recommended know-how information. For example, in the screen of the FIG. 19C, when the management user performs the selection operation of the object, the processing unit 110 performs processing to add the corresponding recommended know-how information to the list information 123 of the corresponding user. In this way, the management user can easily add necessary recommended know-how information while checking the usage and registration status of each working user's know-how information 121. In the FIG. 19, the management user can request adding the recommended know-how information to the working user, but it is not limited to this, for example, the management user may request adding the know-how information retrieved by the management user to the selected working user.

Also, the working users mentioned above are in-hospital users who are responsible for a prescribed patient in the hospital. And the multiple users in this embodiment may include an out-of-hospital users who are in charge of out-of-hospital assistance for the above the prescribed patient. For example, if a prescribed working user is in charge of a prescribed patient in the hospital and another user takes care of the prescribed patient after discharge, the another user becomes an out-of-hospital user.

For example, in the FIG. 19B, two users denoted as “I” (corresponding to Name 1 and Name 2) are the in-hospital users and a user denoted as “O” (corresponding to Name 3) is the out-of-hospital user. The out-of-hospital users provide home care or assistance for patients, for example. When the selection operation of the out-of-hospital user is performed, the know-how information 121 used by the out-of-hospital user, the registered know-how information 121, etc. are displayed as in the FIG. 19C. By making such a display, it is possible to present the usage status of the know-how information 121 to the management user in an easy-to-understand manner, even for an out-of-hospital user who is in charge of the same patient.

If an adverse event occurs in a patient assisted by a prescribed working user, the RN who is manager or the hospital as a whole may be responsible for the adverse event. For example, as mentioned above, the hospital needs to cover medical costs regarding to HACs. And the adverse events here include readmission within a short period of time of the patient's discharge. In the United States, for example, medical fees are reduced when a patient who has been discharged from the hospital is readmitted to the hospital within days for the same disease. In other words, as with HACs, the re-hospitalization in a short period of time is an important issue that should be controlled not only by measures at the level of working users but also by organizational management.

Accordingly, the processing unit 110 may perform processing to present the management user with the second recommended know-how information, which is the know-how information 121 recommended for use by the out-of-hospital user in order to suppress the readmission of the patient. For example, the know-how information 121 may include information about a patient's disease. In this way, the know-how information 121 can be identified to provide appropriate assistance to the patients hospitalized due to a specific disease. The processing unit 110 identifies the second recommended know-how information based on the disease that caused the patient's hospitalization and the information regarding to the disease included in the know-how information 121.

For example, the processing unit 110 determines whether the out-of-hospital user is using the know-how information 121 that contributes to the suppression of re-hospitalization of the patient, based on the patient's disease and the know-how information 121 that the out-of-hospital user is using. For example, the processing unit 110 may display a warning when there is the out-of-hospital user who does not use the know-how information 121 corresponding to the patient's disease. For example, in the example of the FIG. 19B, an object including the text “Warning” is displayed in the area corresponding to the out-of-hospital user. Then, when the selection operation of the out-of-hospital user is performed, the display procedure of the second recommended know-how information recommended to the out-of-hospital user is performed as in RE 7 of the FIG. 19C.

In this way, it is possible to let the management user decide whether or not appropriate assistance is to be provided to prevent readmission of the discharged patients in a short period of time because the assistance status of the out-of-hospital user could be presented to the management user. Similarly to the above example, if the processing unit 110 receives a request adding the second recommended know-how information from the management user, the processing unit 110 may perform processing to add the second recommended know-how information to the list information 123 of the out-of-hospital user without permission from the out-of-hospital user.

In addition, the server system 100 of the present embodiment includes a charging processing unit (not shown). The charging processing unit performs processing to determine the value for the use of the know-how information 121 by the user, and performs processing to request the determined value and settlement processing. By requesting compensation for the use of the know-how information 121 in this way, it becomes possible, for example, to pay compensation to the user who registered the know-how information 121. Motivation to register the know-how information 121 is enhanced, so the tacit knowledge can be efficiently collected.

In this case, since the out-of-hospital user is not a user who works at the hospital, it is assumed that the out-of-hospital user by himself or herself or the direct employer of the out-of-hospital user will pay for the use of know-how information 121. However, if the management user adds the second recommended know-how information to the list information 123 of the out-of-hospital user, the charging processing unit may also charge the costs to the management user or the hospital to which the management user belongs. Since the use of the know-how Information 121 is beneficial for the hospital by preventing readmission, it is considered a necessary expense for the hospital. In this way, by allowing the management user to edit the list information 123 of the out-of-hospital users who are not in a direct hierarchical relationship and paying the costs for doing so, the necessary know-how information 121 can be easily provided to the out-of-hospital users. Patients receive appropriate assistance seamlessly during hospitalization and after discharge, which can reduce readmissions. That is, the method of this embodiment enables the use of a wide range of tacit knowledge, including users outside the hospital, and enables the control of medical expenses, etc.

### 3.6 GPO (Group Purchasing Organization)

The medical facilities may use the GPO (Group Purchasing Organization) to purchase medical devices. The GPO is an industry that specializes in price negotiations with manufacturers and other distributors, and provides members with services to reduce unit prices by committing to purchase large quantities of lots. Even if the minimum purchase lot desired by the manufacturer is pretty large, the GPO enables medical facilities which are member to purchase only as many products with high unit prices as needed while keeping costs down. In the United States, for example, many medical facilities are affiliated with the GPO and purchase various medical devices through the GPO.

The GPO provides terms of purchase (contracts), and members pay the GPO a portion of the purchase price as a fee when they use the contract. The content of the contracts vary, with prices set for each manufacturer and discounts based on the volume of purchases.

Computer systems and methods suitable for GPO are described in U.S. patent application Ser. No. 15/783,992 filed on Oct. 13, 2017 as “COMPUTER-BASED SYSTEMS SPECIFICALLY CONFIGURED TO MANAGE SOFTWARE OBJECTS THAT ARE INTERRELATED VIA TRIGGER CONDITIONS AND METHODS OF USE THEREOF” and U.S. patent application Ser. No. 16/985,609 filed on Aug. 5, 2020 as “METHOD AND SYSTEMS FOR PROVIDING IMPROVED MECHANISM FOR UPDATING HEALTHCARE INFORMATION SYSTEM SYSTEMS.” These patent applications are incorporated by reference herein in their entirety.

In the FIG. 6 in U.S. patent application Ser. No. 15/783,992, a screen example to create an RFP (Request For Proposal) by entering the parameters of the contracts, conditions of the discount, etc., from buyers. In this example, the RFP is created based on a product category designation using a product classification code such as UNSPSC (United Nations Standard Products and Services Code), and the created RFP is transmitted to one or more suppliers.

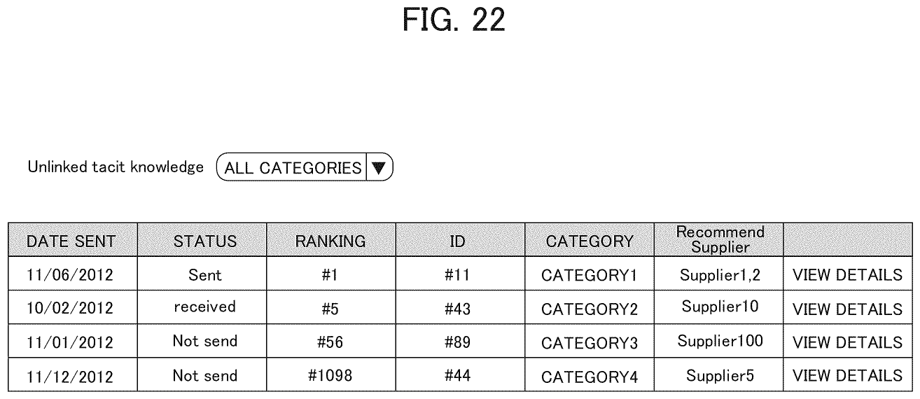

When the supplier responds with a specific product based on the RFP, the buyer is presented with a screen corresponding to, for example, the FIG. 22. The FIG. 22 is a screen that serves as an interface for selecting products, and the FIG. 22 enables the buyers to select products according to their needs while referring to multiple products with each other.

The FIG. 10 in U.S. patent application Ser. No. 16/985,609 also discloses a screen to evaluate the effect of replacing a prescribed product with another.

As can be seen from these descriptions, it is important for the GPO to propose the appropriate products to meet the buyer's requirements. The method of this embodiment may be used for consulting the GPO, specifically for supporting product proposals by the GPO.

For example, if the processing unit 110 associates prescribed know-how information 121 with a plurality of devices as devices, the processing unit 110 may present a device other than the first devices as an alternative device of the first device among the multiple devices.

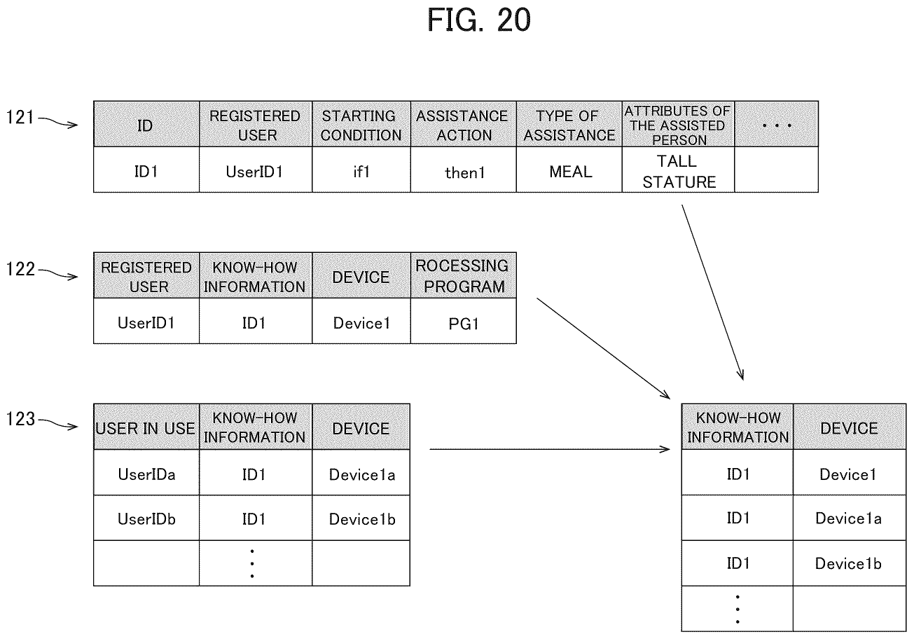

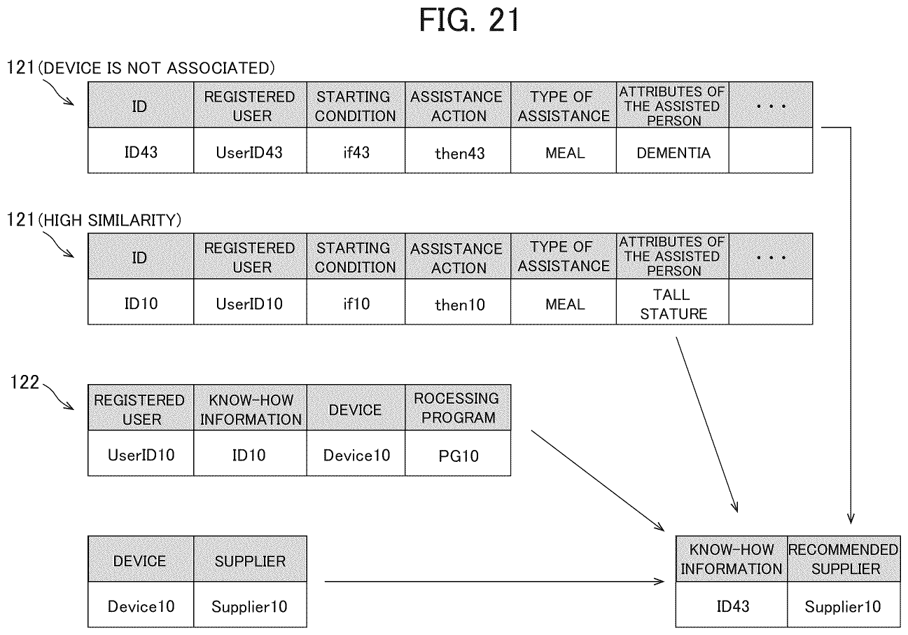

The FIG. 20 is an example of the know-how information 121, the registration information 122, and the list information 123 described above. As described above, the registered user performs the processing shown in the FIG. 6, so that the know-how information 121 is associated with the device for determining either the starting condition of the know-how information 121 or the assistance action. Also, each user performs the processing shown in the FIG. 10 (especially the step S308), so that the know-how information 121 is associated with the device for determining either the starting condition of the know-how information 121 or the assistance action. Thus, as shown in the FIG. 20, one or more devices associated with a prescribed know-how information 121 can be identified. Furthermore, as shown in the FIG. 20, by further using the know-how information 121 itself, it is possible to associate more detailed information with the device, such as specific starting conditions and assistance action

As described above, the Device 1 included in the registration information 122 is the device specified by the registered user to determine the starting conditions, etc. The Device 1a included in the list information 123 is a device specified by another user to use the tacit knowledge of the registered user. That is, since the multiple devices associated with the prescribed know-how information 121 are all devices for determining the same starting conditions, etc., these devices are highly likely to be similar. The same is true for the Device 1b.

Thus, the processing unit 110 may perform processing, for example, if the buyer may consider replacing the Device 1, to propose the Device 1a and the Device 1b as alternative devices. In this way, it is possible to identify and present products that meet the user requirements from a different point of view from product classification codes such as UNSPSC.

In addition, the processing unit 110 may perform processing, for example, if the buyer may consider replacing devices using the prescribed know-how information 121, to propose one or more devices associated with the similar know-how information which is similar to the prescribed know-how information as alternative devices. The similar know-how information is determined based on the first similarity determination processing as described above. The know-how information 121 and the similarity know-how information has a high degree of the similarity, for example, between texts representing starting conditions or between the types of assistance action to be used. Therefore, there is a high probability that know-how information 121 and the similar know-how information are used in similar situations, and it is considered that the device associated with the similar know-how information is also similar to the device to be replaced. By using the similar know-how information in this way, it is possible to increase the number of devices that can be presented and to support a wide range of proposals.

The method of this embodiment need not be fixed to the method of proposing a device using know-how information 121. For example, the processing unit 110 may be able to switch between alternative device determination processing using codes such as UNSPSC and alternative device determination processing using the know-how information 121. For example, the processing unit 110 determines whether to use the codes or the know-how information 121 based on user input.

In addition, the processing unit 110 of the present embodiment may perform processing to identify the fifth know-how information that is associated with devices and has a high degree of similarity to the fourth know-how information to which is not associated with devices, from a plurality of pieces of know-how information 121. The processing unit 110 performs processing to determine the supplier of the device associated with the fifth know-how information as the supplier of the device for determining the starting condition of the fourth know-how information.

The FIG. 21 is a diagram explaining the process of identifying the suppliers based on the fourth know-how information and the fifth know-how information. For example, the know-how information 121 of ID 43 corresponds to the fourth know-how information. The know-how information 121 of ID 43 has no corresponding registration information 122 and is not associated with any devices. The similarity determination unit 113 obtains the similar know-how information similar to the know-how information 121 of ID 43 based on the first similarity determination processing. For example, the know-how information 121 of ID 10 is the similar know-how information and corresponds to the fifth know-how information.

Here, as for the know-how information 121 of ID 10, the corresponding registration information 122 exists as shown in the FIG. 21, and the Device 10 is associated as a device. The storage unit 120 separately stores information that associates the device with the supplier supplying the device. For example, the Device 10 is associated with the supplier 10.

In this case, the processing unit 110 performs processing to propose Supplier 10 as a supplier of devices to be used for the know-how information 121 of ID 43. As described above, since the know-how information 121 of ID 43 and the know-how information 121 of ID 10 are similar, it is highly possible that a device similar to the Device 10 is available for the automatic determination of the know-how information 121 of ID 43. That is, the device used for the automatic determination of the know-how information 121 of ID 43 has a high affinity with the supplier 10, and there is a possibility that the supplier 10 can develop and provide it.

As described above, since no device is associated with the know-how information 121 of ID 43, it is possible that devices suitable for automatic determination of starting conditions and assistance action are not widely available in the market. However, since it was registered as the know-how information 121 and is information that represents the tacit knowledge of any user, it may be useful in the scene of assistance. In this regard, according to the method of this embodiment, the information for automating the processing of the know-how information 121 that could not be handled by existing devices can be presented. As a result, it will be possible to develop new sensing device markets.

The processing unit 110 may use the degree of usage or popularity of each know-how information 121 when determining the fourth know-how information 121. For example, the processing unit 110 may count the number of times downloaded to be used for each know-how information 121. The processing unit 110 may also count the number of users currently using each know-how information 121. The number of downloads and the number of users who use the information shows how many users the targeted know-how information 121 was determined to be useful to. That is, the know-how information 121 with many downloads, etc., may be widely used, and there is a great need for devices that can automate the processing of the know-how information 121. That is, since it is expected that the market size will be somewhat large, it will also motivate the suppliers to enter the market and make it easier for the selected suppliers to actually start supplying devices.

Also, in this embodiment, the know-how information 121 used by each user may be evaluated. There are various aspects of the evaluation, but each user, for example, scores the know-how information 121. The know-how information 121 with high score statistics (such as average values) has high user support and may be widely used. Therefore, in this case as well, there is a great need for devices that can automate the use of know-how information 121.

The FIG. 22 is an example of a screen that presents a recommended supplier. In the example of the FIG. 22, the ID number of the know-how information 121 with which no device is associated and the recommended supplier associated with the know-how information are displayed. The category in the FIG. 22 refers to a category determined based on a product classification code such as UNSPSC. For example, in the example of the FIG. 22, the supplier 1 and the supplier 2 are presented as the suppliers of devices corresponding to know-how information 121 of ID 11. If there are multiple devices associated with the fifth know-how information as shown in the FIG. 20, or if multiple pieces of fifth know-how information similar to one piece of fourth know-how information are selected, there may be multiple recommended suppliers. Similarly, the supplier 10 is presented as a the supplier of devices corresponding to the know-how information 121 of ID 43. The same applies to other know-how information 121. In this way, the recommended supplier for each know-how information 121 can be presented in an easy-to-understand manner.

At this time, the processing unit 110 may present the ranking of each know-how information 121. As mentioned above, the index determining the ranking may be the number of downloads, the number of users, or the evaluation value, or information combined with these data. For example, the know-how information 121 of ID 11 has a high ranking, and if a device for automating this determination is supplied, many users would like to use it. Thus, it is useful to use the ranking for processing because it is a material that encourages suppliers to supply new devices. For example, the FIG. 22 is a screen that displays the know-how information 121 that satisfies a prescribed condition among a plurality of know-how information 121 with which no device is associated, in ranking order.

### 4. Modification

### <Data Format>

In this embodiment described above, the know-how information 121, the registration information 122, the list information 123, the facility list information 124, etc. are exemplified as data to be stored in the storage unit 120. However, the data format used in this embodiment is not limited to the above, and various modifications can be performed. For example, data described in multiple tables may be combined into one table. Data described as a single table may also be divided into multiple tables. Also, elements contained in a prescribed table can be added, omitted, or stored as elements of other tables. In the present embodiment, various tables have been used for the explanation, but instead of storing these tables in the storage unit, machine learning such as NN, for example, may be used to determine the correspondence of each element included in the table.

For example, in the above, the registration information 122 is data related to the registered users, and the list information 123 is data when users other than the registered users use the know-how information 121. However, as can be seen from the FIGS. 8 and 11, the data formats are similar, so there is no need to separate them. For example, the list information 123 may be information including the know-how information 121 registered by the target user and the know-how information 121 in use by the target user. For example, when registering the know-how information 121, the registration processing unit 111 may store information identifying the know-how information, device, processing program, etc., in the list information 123 of the registered user.

In addition, an example is explained that the list information 123 is a set of the know-how information 121 currently in use. However, the list information 123 may include the know-how information 121 that has been used but is not currently used. For example, as the status of each know-how information 121 included in the list information 123, information such as downloaded but unused, used, or used in the past but not currently used may be stored. For each know-how information 121, information such as the starting date and time of use, the duration of use, the number of times of use, and the evaluation score may be stored.

In the above, an example is explained that the facility list information 124 refers to a set of the know-how information 121 registered by the belonged user. However, the facility list information 124 may include the know-how information 121 in use by the belonged user.

### <How to Use the Know-How Information 121>

In addition, an example of individual use of the know-how information 121 has been described above. For example, suppose that the P th know-how information associated with the starting condition “if_p” and the assistance action “then_p” and the Q th know-how information associated with the starting condition “if_q” and the assistance action “then_q” are stored. The Device-p is associated with the P th know-how information 121, and the Device-q is associated with the Q th know-how information 121. In this case, in the above example, the processing of presenting “then_p” when it is determined that “if_p” is satisfied using the “Device-p” and the processing of presenting “then_q” when it is determined that “if_q” is satisfied using the “Device-q” are performed independently.

However, the processing of this embodiment is not limited to this. For example, if the P th know-how information is similar to the Q th know-how information, “if_p” and “then_q” may have some relevance. Similarly, “if_q” and “then_p” may have some relevance. Therefore, rather than processing these independently, compound processing may be performed.

For example, the processing unit 110 accepts the sensor information from the Device_p and the sensor information from the Device_q as inputs, and may determine whether “if_p” is satisfied and whether “if_q” is satisfied on the basis of both inputs. For example, the machine learning of the NN with two inputs and two outputs is performed using both the sample data and correct answer data collected for the P th know-how information and the sample data and correct answer data collected for the Q th know-how information. However, the second processing algorithm can be modified in various ways as described above.

In this way, the starting condition and assistance action can be determined after considering the relationship between the know-how information 121, thereby improving the accuracy of processing. Although an example of combining 2 pieces of the know-how information 121 has been described here, complex processing may be performed for 3 or more pieces of the know-how information 121.

### <Third Similarity Determination Processing>

In the above, an example in which the third similarity determination processing is performed according to the FIG. 15 in the same manner as the second similarity determination processing is performed has been described. That is, each of the multiple pieces of the know-how information 121 included in the facility list information 124 is subject to the first similarity determination processing with the know-how information 121 included in the list information 123. However, the specific example of the third similarity determination processing is not limited to this.

For example, the facility list information 124 for a prescribed facility may include information on the number of downloads and the number of users of each know-how information. Then, the similarity determination unit 113 may perform the third similarity determination processing on the basis of a part of the know-how information 121 with a high ranking determined by the number of downloads, etc., among the multiple know-how information 121 included in the facility list information 124. For example, the similarity determination unit 113 makes the prescribed number of top-ranked know-how information 121 among the plurality of know-how information 121 included in the facility list information 124 the object of the third similarity determination processing.

In this way, it becomes possible to limit the target of the third similarity determination processing to the more important information among the multiple know-how information 121 included in the facility list information 124. As a result, it is possible to more accurately determine the compatibility between users and facilities.

### <Recommended Facilities Suitable for Assisted Persons>

In the above, we have explained an example of using the third similarity between the list information 123 of users whose assistance contents have been evaluated and the facility list information 124 to determine a recommended facility suitable for the prescribed assisted person.

However, the processing of this embodiment is not limited to this. The third similarity is an indicator of the point of view of whether or not the method of assistance of the facility is suitable for the target assisted person. However, the degree of satisfaction of the assisted person upon moving into a facility is not determined solely by the method in which he or she is assisted, and various factors such as the degree of progression of dementia of the assisted person and the degree of ADL may influence the degree of satisfaction. For example, if the assisted person moves into a facility and his or her ADL level is relatively low compared to that of other assisted person, the assisted person may not be able to keep up with recreation in the facility, resulting in reduced motivation and the assisted person leaving the facility.

Therefore, the processing unit 110 may perform processing to determine recommended facilities on the basis of information other than the list information 123 and the facility list information 124. For example, inputs of the processing include the list information 123 of users whose assistance content is being evaluated, the facility list information 124 of facilities to be determined, the dementia level information, and ADL evaluating values. The output of the processing is information representing the degree to which the assisted person is suitable for the target facility.

Note that the dementia level information here indicates the degree of progression of dementia in the assisted person. For example, the dementia level information may be a score of the MMSE (Mini-Mental State Examination), a score of the revised Hasegawa's Brief Intelligence Scale (HDS-R), or other information that represents the results of a dementia test. The dementia level information may be information based on brain images obtained using computed tomography (CT) or magnetic resonance imaging (MRI). For example, the dementia level information may be information representing the results of a doctor's diagnosis based on a brain image, the brain image itself, or the result of some image processing on the brain image.

The processing unit 110 may perform processing to determine recommended facilities using machine learning such as the NN. For example, in the learning stage, the storage unit 120 associates the list information, the facility list information, the dementia level information and the ADL evaluating values with information representing the result when the assisted person moves into a prescribed facility as correct data.

The correct data here may be information representing, for example, whether the assisted person continued to live in the target facility or the assisted person left soon. For example, the correct data may be binary data distinguishing between the two, or numerical data representing the duration of continued occupancy.

Alternatively the correct data may be information representing the degree to which the assisted person smiles while living in the facility. The information representing for example, the ratio of the number of times, frequency and time a person smiles while living in a facility to the number of times, frequency and time a person smiles under normal conditions may be calculated as the degree of smiling. The normal conditions may be, for example, conditions the assisted person is at home or conditions being assisted by a user whose assistance content has been evaluated.

Based on the above input data and the correct data, the processing unit 110 performs the machine learning. For example, the processing unit 110 inputs the list information, the facility list information, the dementia level information, and the ADL evaluating values into the NN, and performs forward operations based on the weights at that time. Then, the processing unit 110 calculates the objective function based on the calculation result and the correct data (occupancy condition or degree of smiling), and updates the weights based on the objective function.

In this way, based on the input data, the learned model is generated to obtain an estimate of the occupancy condition of the assisted person when the assisted person lives in the target facility or an estimate of the degree of smiling. The processing unit 110 of this embodiment may calculate the recommended facilities for the assisted person based on the learned model. In this way, it is possible to determine the compatibility between the assisted person and the facility from various points of view as well as the method of assistance.

### <Displaying Recommended User>

In the above, the process of determining a recommended user suitable for assisting a prescribed assisted person based on the second similarity determination processing and the process of determining a recommended user suitable for a prescribed facility based on the third similarity determination processing are explained. An example screen for displaying the information of the recommended user is described below.

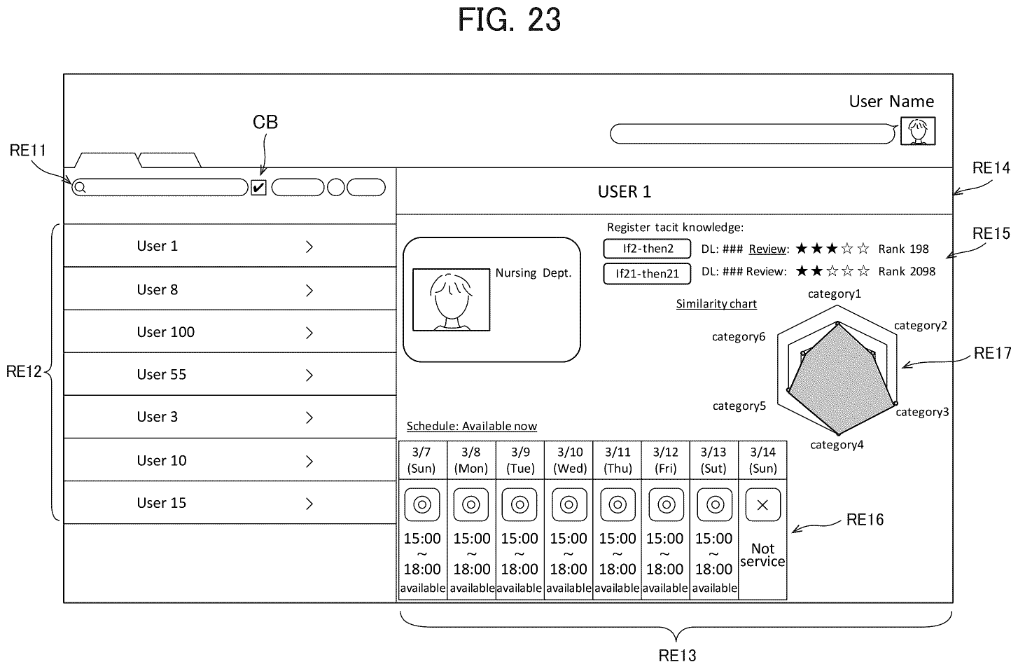

The FIG. 23 is a specific screen example displaying the information of the recommended user. As shown in the FIG. 23, the display screen in this case includes an area RE 11 for entering search keys, a check box CB for switching a similar function on or off, an area RE 12 for displaying one or more recommended users that are search results, and an area RE 13 for displaying detailed information of the recommended users selected in RE 12.

The user performing the search first may input the search key to search for the recommended user into the RE 11. For example, as described above, the user inputs the user ID of the user whose assistance content for the assisted person is evaluated as a search key, and checks the CB. If the CB is checked, the processing unit 110 determines a recommended user based on the second similarity determination processing for determining the similarity between users. That is, in this case, the user who is determined to be similar to the user whose assistance content for the assisted person is evaluated is displayed in the RE 12 as the recommended user. The RE 12 is an area that displays information on a prescribed number of recommended users, for example, in order of increasing similarity.

In this embodiment, the recommended user may be retrieved from a different point of view from the second similarity determination processing. For example, the user may uncheck the CB and input information such as the type of assistance, such as the meal assistance or the excretion assistance, the residence of the assisted person, and the amount of compensation as the search keys. In this case, the processing unit 110 performs processing to display on the RE 12 as a recommended user a user who is good at a prescribed type of assistance, a user who is active in the neighborhood, a user who accepts a request for a fee less than the input amount, etc.

The RE 13 includes an area RE 14 for displaying user names, an area RE 15 for displaying the registered know-how information, an area RE 16 for displaying the schedules, and an area RE 17 for displaying similarity by category.

The know-how information 121 displayed in the RE 15 is determined based on the registration information 122. As shown in the FIG. 23, in each know-how information 121, the number of downloads, review results by other users, rankings, etc., are displayed. In this way, the user performing the search can be presented with how useful tacit knowledge the target user holds.

The RE 16 is an area that displays information indicating whether the target user can provide assistance services on a daily basis. In the example in the FIG. 23, the target user can provide assistance services from 3/7 to 3/13, but can not provide assistance services on 3/14. Also, as shown in the FIG. 23, the service availability period may be displayed for each day. In this way, the user who performed the search could decide when to request assistance while looking at the user's or the assisted person's schedule and the recommended user's schedule.

For each category of assistance, the RE 17 includes an object that displays the similarity between the user or facility identified by the search key and the recommended user. For example, the categories 1 to 6 in the FIG. 23 present the types of assistance, such as the meal assistance, the excretion assistance, and the transferring or moving assistance, respectively. For example, the similarity determination unit 113 may classify the multiple pieces of know-how information 121 included in the list information 123 by category based on the additional information of the know-how information 121. Then, the similarity determination unit 113 calculates, for each category, the second similarity between the list information 123 of the user corresponding to the search key and the list information 123 of the other user. In the example of the FIG. 23, the second similarity determination processing is performed for each of the six categories to obtain six the second similarities, and the values of six the second similarities are displayed as graphs. Different types of assistance are needed depending on the situation of the assisted person and their family members, for example, requesting the meal assistance but not much transferring or moving assistance. In this regard, by presenting the degree of similarity for each category to the user as shown in the FIG. 23, it becomes possible to more easily present whether or not the recommended user is suitable for assisting the assisted person. In the example of the FIG. 23, if a person tries to request assistance corresponding to a category 3 or 4, the corresponding user is more likely to be selected, and if a person tries to request assistance corresponding to a category 2 or 6, the corresponding user is less likely to be selected.

Although the present embodiment has been described in detail as described above, it will be easy for those skilled in the art to understand that many modifications are possible that do not materially deviate from the novel matters and effects of the present embodiment. Therefore, all such variations shall be included in the scope of this disclosure. For example, a term appearing at least once in a description or drawing with a different term that is more broadly or synonymously may be replaced by that different term anywhere in the description or drawing. All combinations of this embodiment and variations are also included in the scope of this disclosure. Moreover, the configuration and operation of the information processing system, the information processing device, the server system, the terminal device, etc., are not limited to those described in this embodiment, and various modifications can be performed.

1. An information processing device comprising:
a processing unit configured to receive a registration request of a know-how information including information which associates condition information representing a starting condition and assistance information representing an assistance action to be performed if the starting condition is satisfied; and
a storage unit configured to store a plurality of the know-how information based on a plurality of the registration requests,
wherein the processing unit is configured to output one of the plurality of the know-how information as a search result based on a search request including information to identify either one of the starting condition and the assistance action.

2. The information processing device according to the claim 1, wherein
the condition information is a text representing the starting condition, and
the processing unit is configured to perform processing to identify a device used for determining the starting condition represented by the condition information by performing text analysis processing of the condition information and to associate an information representing the device which is identified with the know-how information.

3. The information processing device according to the claim 2, wherein
the processing unit is configured to perform processing to collect a plurality of device data obtained by the device, and perform processing to transmit addition request to add a correct tag for each of the plurality of device data to a terminal device used by a registered user, the registered user being a user who sent the registration request of the know-how information, and
the correct tag represents determination result by the registered user, whether each of the plurality of device data is satisfied with the starting condition.

4. The information processing device according to claim 1, wherein
the processing unit is capable of receiving a usage request of any of the plurality of the know-how information stored in the storage unit from users, and
the storage unit is configured to store list information including one or more of the know-how information in use in association with each of the users.

5. The information processing device according to the claim 4, wherein
the plurality of the know-how information include a first know-how information, a second know-how information and a third know-how information,
the processing unit is configured to perform similarity determination processing for determining the degree of similarity between any two of the plurality of the know-how information stored in the storage unit, and
in the similarity determination processing,
if a number of the users whose list information includes the first know-how information and the second know-how information is larger than that of the users whose list information includes the first know-how information and the third know-how information, the similarity between the first know-how information and the second know-how information is determined to be higher than the similarity between the first know-how information and the third know-how information.

6. The information processing device according to the claim 4, wherein
the processing unit is configured to perform second similarity determination processing to determine the similarity of the list information corresponding to a first user among the users and the list information corresponding to a second user different from the first user.

7. The information processing device according to the claim 6, wherein
the processing unit is configured to perform the second similarity determination processing on the basis of the list information corresponding to a user who has experience of assisting a given assisted person among the users and the list information of other users among the users, and to perform processing to determine a recommended user recommended for assisting the given assisted person from the other users based on the result of the second similarity determination processing.

8. The information processing device according to claim 4, wherein
the storage unit is configured to store facility list information for each of a plurality of facilities, the list information being a set of the know-how information registered by one or more users who belong to a facility, and
the processing unit is configured to perform third similarity determination processing for determining the similarity between the list information and the facility list information and to determine a recommended facility recommended for a given user or a recommended user recommended for a given facility based on the result of the third similarity determination processing.

9. The information processing device according to claim 4, wherein
the know-how information includes information representing hospital-acquired conditions,
the users include a working user managed by a management user, and
the processing unit is configured to perform processing to determine the recommended know-how information to be used by the working user from the plurality of the know-how information on the basis of occurrence status of the hospital-acquired conditions of the working user and to present the recommended know-how information to the management user.

10. The information processing device according to the claim 9, wherein
the processing unit is configured to perform processing to add the recommended know-how information to the list information of the working user if receiving a request to add the recommended know-how information from the management user.

11. The information processing device according to claim 9, wherein
the working user is an in-hospital user in charge of a patient in a hospital,
the users include an out-of-hospital user who is responsible for the out-of-hospital care of the patient, and
the processing unit is configured to perform processing to present the management user with second recommended know-how information, the second know-how information being the know-how information recommended for use by the out-of-hospital user in order to reduce a readmission of the patient.

12. The information processing device according to the claim 2, wherein
when a plurality of devices are associated with a given know-how information as the device, the processing unit is configured to perform processing to present a device other than a first device among the plurality of devices as an alternative device to the first device.

13. The information processing device according to the claim 2, wherein
the processing unit is configured to perform processing to identify fifth know-how information that the device is associated with and has a high degree of similarity to fourth know-how information to which the device is not associated, and to perform processing to determine a supplier of the device associated with the fifth know-how information, as a supplier of the device for determining the starting condition of the fourth know-how information.

14. An information processing method comprising:
receiving a registration request of a know-how information including information which associates condition information representing a starting condition and assistance information representing an assistance action to be performed if the starting condition is satisfied; and
outputting one of a plurality of the know-how information stored by a plurality of the registration requests as a search result based on a search request including information to identify either one of the starting condition and the assistance actions.

15. The information processing device according to claim 2, wherein
the processing unit is capable of receiving a usage request of any of the plurality of the know-how information stored in the storage unit from users, and
the storage unit is configured to store list information including one or more of the know-how information in use in association with each of the users.

16. The information processing device according to claim 3, wherein
the processing unit is capable of receiving a usage request of any of the plurality of the know-how information stored in the storage unit from users, and
the storage unit is configured to store list information including one or more of the know-how information in use in association with each of the users.

17. The information processing device according to the claim 5, wherein
the processing unit is configured to perform second similarity determination processing to determine the similarity of the list information corresponding to a first user among the users and the list information corresponding to a second user different from the first user.

18. The information processing device according to claim 5, wherein
the storage unit is configured to store facility list information for each of a plurality of facilities, the list information being a set of the know-how information registered by one or more users who belong to a facility, and
the processing unit is configured to perform third similarity determination processing for determining the similarity between the list information and the facility list information and to determine a recommended facility recommended for a given user or a recommended user recommended for a given facility based on the result of the third similarity determination processing.

19. The information processing device according to claim 6, wherein
the storage unit is configured to store facility list information for each of a plurality of facilities, the list information being a set of the know-how information registered by one or more users who belong to a facility, and
the processing unit is configured to perform third similarity determination processing for determining the similarity between the list information and the facility list information and to determine a recommended facility recommended for a given user or a recommended user recommended for a given facility based on the result of the third similarity determination processing.

20. The information processing device according to claim 7, wherein
the storage unit is configured to store facility list information for each of a plurality of facilities, the list information being a set of the know-how information registered by one or more users who belong to a facility, and
the processing unit is configured to perform third similarity determination processing for determining the similarity between the list information and the facility list information and to determine a recommended facility recommended for a given user or a recommended user recommended for a given facility based on the result of the third similarity determination processing.
